(19) United States
(12) Patent Application Publication
(10) Pub. No.: US 2014/0052264 A1
(43) Pub. Date: Feb. 20, 2014

(54) Porous, Stabilized Craniomaxillofacial Implants and Methods and Kits Relating Thereto

(71) Applicant: Ticona LLC, Florence, KY (US)

(72) Inventors: Julia Hufen, Rheinberg (DE); Anthony Verrocchi, Covington, KY (US); Rainer Walkenhorst, Melle (DE)

(73) Assignee: Ticona LLC, Florence, KY (US)

(21) Appl. No.: 13/964,756

(22) Filed: Aug. 12, 2013

Related U.S. Application Data
(60) Provisional application No. 61/684,937, filed on Aug. 20, 2012.

(51) Int. Cl. A61F 2/28

(52) U.S. Cl. CPC A61F 2/2803
USPC 623/17.17; 264/118

(57) ABSTRACT

Porous craniomaxillofacial implants may include a plurality of polymeric particles sintered together at a plurality of contact points to form a porous network having pores; and an antioxidant disposed on a surface of at least some of the polymeric particles and/or in the pore of the porous network, so as to stabilize the implants from at least some oxidative damage during storage, transportation, and/or implementation (in vivo and/or ex vivo). Preferably, the polymeric particles include at least one of polyethylene, a polyethylene-polypropylene copolymer, a polyethylene-polybutylene copolymer, polytetrafluoroethylene, and expanded polytetrafluoroethylene.

### BACKGROUND

The present invention relates to porous craniomaxillofacial implants stabilized with antioxidants, and methods and kits relating thereto.

Surgeons often repair or augment bone structures in reconstructive surgeries that may be necessitated by injury or birth defects. While natural bone from the patient is a preferred material for repairing bone structure, in craniomaxillofacial repair and augmentation, natural bone may not be practical. As it is not easy to reshape bone, the bone graft is often matched in size and shape to the area that needs to be repaired. Finding such a match is often difficult, and sometimes impossible, given the contours of the various areas of the craniomaxillofacial area. Accordingly, a craniomaxillofacial bone graft can leave deformed features that could require further surgery to repair.

Synthetic materials have been used as an alternative to bone grafts. One of the more common synthetic materials in craniomaxillofacial repair and augmentation is high-density polyethylene, and is often utilized as a porous structure having high surface area to allow for fibroblast intergrowth.

The advantages often touted in regards to the use of ultrahigh molecular weight polyethylene include the chemical stability, no donor site morbidity, and availability. However, recent studies have shown that after implantation polyethylene slowly undergoes oxidation at the surface, which causes the polyethylene to become brittle, which can lead to wear of the implants, and abrasion of surrounding tissue and/or bone. Consequently, in some instances, the implant may need to be replaced, which can involve additional surgeries with potential complications. The consequences of polyethylene oxidation may be magnified in craniomaxillofacial implants as the high surface area of the porous structure provides for a greater percentage of the implant to become brittle.

### SUMMARY OF THE INVENTION

The present invention relates to porous craniomaxillofacial implants stabilized with antioxidants, and methods and kits relating thereto.

One embodiment of the present invention may provide for a porous implant that comprises a plurality of polymeric particles sintered together at a plurality of contact points to form a porous network having pores, the plurality of polymeric particles comprising polyethylene; and an antioxidant disposed on a surface of at least some of the polymeric particles and/or in the pore of the porous network.

Another embodiment of the present invention may provide for a porous implant that comprises a plurality of polymeric particles sintered together at a plurality of contact points to form a porous network having pores, the plurality of polymeric particles comprising at least one polymer selected from the group consisting of a polyethylene-polypropylene copolymer, a polyethylene-polybutylene copolymer, polytetrafluoroethylene, and expanded polytetrafluoroethylene; and an antioxidant disposed on a surface of at least some of the polymeric particles and/or in the pore of the porous network.

Another embodiment of the present invention may provide for a method of forming a porous implant, the method comprising heating a plurality of polymeric particles in a mold so as to sinter together the polymeric particles at a plurality of contact points thereby forming a porous network, the plurality of polymeric particles comprising polyethylene; and contacting the polymeric particles with an antioxidant.

Yet another embodiment of the present invention may provide for a method of forming a porous implant, the method comprising heating a plurality of polymeric particles in a mold so as to sinter together the polymeric particles at a plurality of contact points thereby forming a porous network, the plurality of polymeric particles comprising at least one polymer selected from the group consisting of a polyethylene-polypropylene copolymer, a polyethylene-polybutylene copolymer, polytetrafluoroethylene, and expanded polytetrafluoroethylene; and contacting the polymeric particles with an antioxidant.

Another embodiment of the present invention may provide for a method for structurally modifying a portion of a cranium, the method comprising implanting a porous surgical implant into a cranium, the porous surgical implant comprising: a porous structure formed from a plurality of polymeric particles sintered at a plurality of contact points, the plurality of polymeric particles comprising polyethylene, and an antioxidant.

Another embodiment of the present invention may provide for a method for structurally modifying a portion of a cranium, the method comprising implanting a porous surgical implant into a cranium, the porous surgical implant comprising: a porous structure formed from a plurality of polymeric particles sintered at a plurality of contact points, the plurality of polymeric particles comprising at least one polymer selected from the group consisting of a polyethylene-polypropylene copolymer, a polyethylene-polybutylene copolymer, polytetrafluoroethylene, and expanded polytetrafluoroethylene, and an antioxidant.

The features and advantages of the present invention will be readily apparent to those skilled in the art upon a reading of the description of the preferred embodiments that follows.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIGS. 1A-B provide illustrative scanning electron micrographs of polyethylene particles described herein having popcorn shapes and potato shapes, respectively.

FIG. 2 provides an illustrative scanning electron micrograph of polyethylene particles bound together where the polyethylene particles have a surface roughness that is about nano-rough to about micro-rough.

FIG. 3A illustrates an example of a porous implant of the present invention being placed in a cranium as a cranial implant.

FIG. 3B illustrates a more detailed view of a cranial implant of 3A including an adhesive.

FIG. 4A illustrates an example of a porous implant of the present invention as an orbital reconstruction implant.

FIG. 4B illustrates a cross-section of a orbital reconstruction implant FIG. 4B.

FIG. 4C illustrates an example of a reinforcing mesh that includes securing components.

FIG. 5 illustrates an example of a porous implant of the present invention being placed as a mandible implant.

### DETAILED DESCRIPTION

The present invention relates to porous craniomaxillofacial implants stabilized with antioxidants, and methods and kits relating thereto.

The present invention provides for, in some embodiments, porous implants that comprise a plurality of polymeric particles sintered together at a plurality of contact points so as to form a porous network and an antioxidant adsorbed to the surface of at least a portion of the polymeric particles. As used herein, the term “sintered,” and the like, refers to a physical connection that bonds two particles together. The bonds between two sintered particles may be rigid or flexible depending on at least the composition of the polymeric particles. The bonding may or may not also involve chemical bonding. In preferable embodiments, a polymeric particulate may comprise at least one polymer selected from the group of polyethylene, polyethylene-polypropylene copolymers, polyethylene-polybutylene copolymers, polytetrafluoroethylene, or expanded polytetrafluoroethylene.

Without being limited by theory, it is believed that the antioxidant may mitigate and/or slow the oxidation of the polymeric particles of the implant, thereby extending the lifetime of the implant.

Further, the porous network of the porous implant may enable infiltration of a patient's cells into the porous implant, which may lead to vascularization and tissue growth in and around the porous implant, and consequently mitigate implant rejection by the patient.

It should be noted that when “about” is provided herein at the beginning of a numerical list, “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit.

Porous implants of the present invention may, in some embodiments, comprise a plurality of polymeric particles sintered together at a plurality of contact points so as to form a porous network, and an antioxidant adsorbed to a surface of at least a portion of the polymeric particles. It should be noted that if the antioxidant is relatively solid, it will most likely be present on the surface of at least a portion of the polymeric particles. On the other hand, if the antioxidant is a liquid, it also is on the surface of at least a portion of the polymeric particles, but can diffuse over time through the pores of the porous network. Thus, in some embodiments, porous implants of the present invention may comprise antioxidants adsorbed to the surface of at least some of the polymeric particles and also antioxidants disposed in the pores of the porous network.

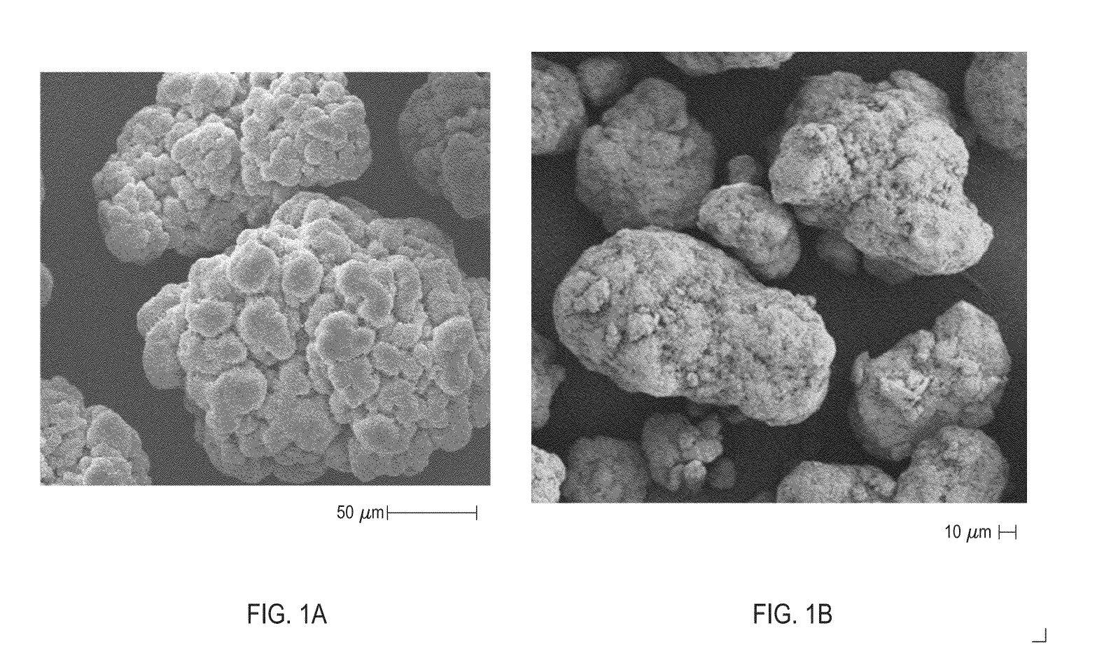

It should be understood that as used herein, the terms “particle” and “particulate” may be used interchangeably and include all known shapes of materials, including spherical, ovular, substantially spherical, substantially ovular, prolate spheroidal, popcorn, substantially popcorn, potato, substantially potato, discus, platelet, flake, ligamental, acicular, fibrous, polygonal (such as cubic), randomly shaped (such as the shape of crushed rocks), faceted (such as the shape of crystals), and any hybrid thereof, and the like with rounded corners and/or edges. Illustrative examples of popcorn shapes and potato shapes are provided in FIGS. 1A-B, respectively.

In preferable embodiments, a polymeric particulate may comprise at least one polymer selected from the group of polyethylene, polyethylene-polypropylene copolymers, polyethylene-polybutylene copolymers, polytetrafluoroethylene, or expanded polytetrafluoroethylene.

Polymeric particles suitable for use in conjunction with porous implants of the present invention may comprise high to ultrahigh molecular weight polymers of the compositions described herein. As used herein, the use of “high to ultrahigh molecular weight polymer” should be taken to encompass high molecular weight polymer, very-high molecular weight polymer, ultrahigh molecular weight polymer, and any blend thereof. As used herein, the term “high molecular weight polymer” refers to a polymer composition having an average molecular weight of about 300,000 g/mol to about 1,000,000 g/mol. As used herein, the term “very-high molecular weight polymer” refers to a polymer composition having an average molecular weight of about 1,000,000 g/mol to about 3,000,000 g/mol. As used herein, the term “ultrahigh molecular weight polymer” refers to a polymer composition having an average molecular weight of about 3,000,000 g/mol to about 20,000,000 g/mol.

In some embodiments, polymeric particles suitable for use in conjunction with porous implants of the present invention may comprise high to ultrahigh molecular weight polymer having an average molecular weight ranging from a lower limit of about 300,000 g/mol, 500,000 g/mol, 1,000,000 g/mol, 2,000,000 g/mol, 3,000,000 g/mol, or 5,000,000 g/mol to an upper limit of about 20,000,000 g/mol, 15,000,000 g/mol, 12,000,000 g/mol, 10,000,000 g/mol, 9,000,000 g/mol, 5,000,000 g/mol, 3,000,000 g/mol, or 1,000,000 g/mol, and wherein the average molecular weight may range from any lower limit to any upper limit and encompass any range therebetween. Methods suitable for measuring the molecular weight of polymers described herein include ASTM D4020.

Polyethylene particles suitable for use in conjunction with porous implants of the present invention may, in some embodiments, include, but are not limited to, commercially available GUR® (ultrahigh molecular weight polyethylene, available from Ticona Polymers LLC). Specifically, GUR® polymers may include: GUR® 1020, GUR® 1050, GUR® 2000 series (2105, 2122, 2122-5, 2126), GUR® 4000 series (4113, 4120, 4130, 4150, 4152 4170, 4012, 4122-5, 4022-6, 4050-3/4150-3), GUR 8000® series (8110, 8020), GUR® X series (X143, X184, X168, X172, X192, X198), and the like, any derivative thereof, and any combination thereof. These are all available from Ticona Polymers LLC.

Polytetrafluoroethylene particles suitable for use in conjunction with porous implants of the present invention may, in some embodiments, include, but are not limited to, commercially available POLYFLON™ and POLYFLON™ M-Series (available from Daikin), TEFLON® (available from DuPont), DYNEON™ PTFE (available from 3M), ALGOFLON® (available from Solvay), and the like, any derivative thereof, and any combination thereof.

Polypropylene particles suitable for use in conjunction with porous implants of the present invention may, in some embodiments, include, but are not limited to, commercially available ExxonMobil™ polypropylene resin (available from ExxonMobil), PURELL™ (available from LyondellBasell), MOPLEN® (available from LyondellBasell), SABIC® PP (available from Saudi Basic Industries Corporation), and the like, any derivative thereof, and any combination thereof.

In some embodiments, the polymeric particles suitable for use in conjunction with porous implants of the present invention may have a diameter in at least one dimension ranging from a lower limit of about 10 microns, 50 microns, 100 microns, 150 microns, 200 microns, and 250 microns to an upper limit of about 5000 microns, 2000 microns, 1000 microns, 750 microns, 500 microns, 400 microns, 300 microns, 250 microns, 200 microns, 150 microns, or 100 microns, and wherein the diameter in at least one dimension may range from any lower limit to any upper limit and encompass any range therebetween.

In some embodiments, the polymeric particles suitable for use in conjunction with porous implants of the present invention may have a bulk density ranging from a lower limit of about 0.10 g/cm³, 0.25 g/cm³, or 0.5 g/cm³ to an upper limit of about 0.9 g/cm³, 0.75 g/cm³, or 0.5 g/cm³, and wherein the bulk density may range from any lower limit to any upper limit and encompass any range therebetween.

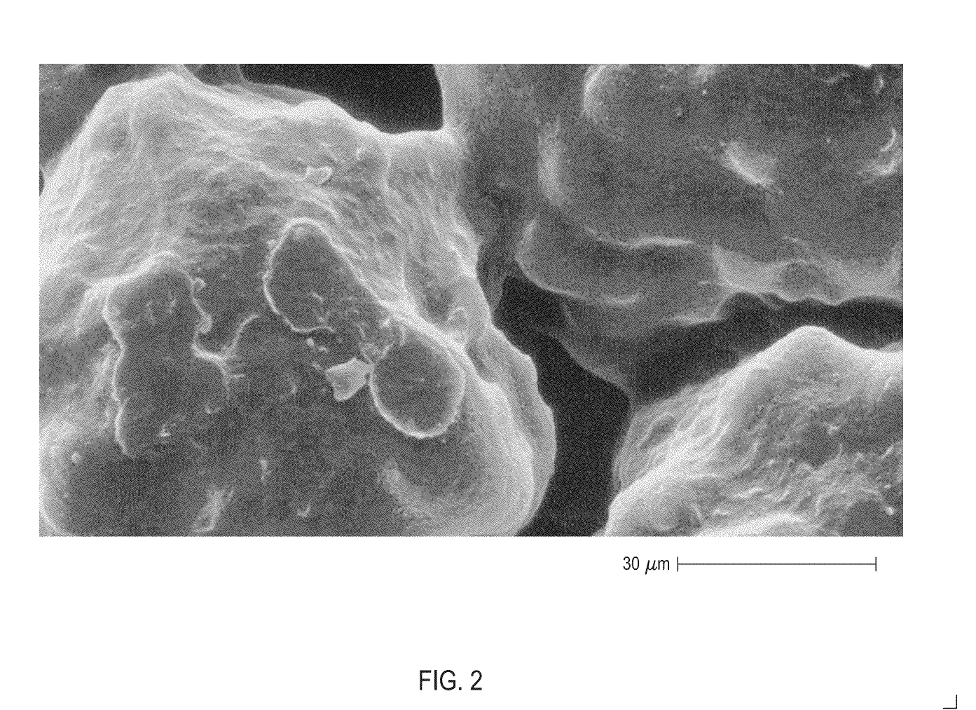

In some embodiments, the polymeric particles suitable for use in conjunction with porous implants of the present invention may have a surface roughness that is about nano-rough to about micro-rough. As used herein the term “micro-rough” refers to a surface having features that extend about 0.5 microns to about 20 microns from the surface. As used herein, the term “nano-rough” refers to the surface having features that extend about 1 nm to about 500 nm from the surface. FIG. 2 provides a nonlimiting illustration of polyethylene particles bound together where the polyethylene particles have a surface roughness that is about nano-rough to about micro-rough.

Without being limited by theory, it is believed that surface roughness may influence the ability for cells to grow on the surface of the polymeric particles, and consequently infiltrate the porous network of a porous implant to improve implantation success. Further, it is believed that nano-rough to micro-rough surfaces may enhance cell growth on polymeric particles, thereby enhancing the biocompatibility and mitigate patient rejection of the porous implants of the present invention. It should be noted that surface roughness is preferably determined after formation of the porous implant as some embodiments of forming porous implants, as described herein, include temperature and pressure changes that may alter the surface roughness of the polymeric particles.

In some embodiments, the polymeric particles suitable for use in conjunction with porous implants of the present invention may have a surface roughness with features extending from the surface in an amount ranging from a lower limit of about 1 nm, 10 nm, 100 nm, 250 nm, 500 nm, 1 micron, or 2 microns to an upper limit of about 20 microns, 15 microns, 10 microns, 5 microns, 2 microns, 1 micron, 500 nm, or 250 nm, and wherein the surface features may extend in an amount range from any lower limit to any upper limit and encompass any range therebetween.

In some embodiments, a porous implant of the present invention may comprise a mixture of two or more polymeric particles distinguished by at least one of: composition, particle size, shape, morphology, additivation, aspect ratio, bulk density, surface roughness, and any combination thereof. By way of nonlimiting example, a porous implant of the present invention may comprise a plurality of polymeric particles having a multimodal diameter distribution. For example, a bimodal diameter distribution derived from a mixture of a first plurality of polymeric particles having an average diameter of about 50 microns and a second plurality of polymeric particles having an average diameter of 500 microns.

By way of another nonlimiting example, a porous implant of the present invention may comprise a first plurality of polymeric particles comprising high molecular weight polymer and a second plurality of polymeric particles comprising ultrahigh molecular weight polymer. By way of yet another nonlimiting example, a porous implant of the present invention may comprise a first plurality of polymeric particles comprising high molecular weight polymer having a substantially potato shape and a second plurality of polymeric particles comprising ultrahigh molecular weight polymer having a substantially popcorn shape.

By way of another nonlimiting example, a porous implant of the present invention may comprise a first plurality of polytetrafluoroethylene particles and a second plurality of polyethylene particles. By way of yet another nonlimiting example, a porous implant of the present invention may comprise a first plurality of polytetrafluoroethylene particles and a second plurality of expanded polytetrafluoroethylene particles. By way of another nonlimiting example, a porous implant of the present invention may comprise a first plurality of polyethylene particles and a second plurality of expanded polytetrafluoroethylene particles. By way of another nonlimiting example, a porous implant of the present invention may comprise a first plurality of polyethylene particles, a second plurality of polytetrafluoroethylene particles, and a third plurality of expanded polytetrafluoroethylene particles. It should be noted that each plurality of particle may, in some embodiments, differ from each other in more than their composition, e.g., particle size, roughness, and the like described herein.

In some embodiments, the porous network of a porous implant of the present invention may comprise a plurality of pores. The dimensions of such pores may depend on, inter alia, the size of the polymeric particles, the size of the fusions between particles, the interstitial spaces between particles, and any additives included in the porous implant. In some embodiments, pores of a porous network described herein may have an average diameter ranging from a lower limit of about 15 microns, 25 microns, 50 microns, 100 microns, 150 microns, 200 microns, or 300 microns to an upper limit of about 500 microns, 400 microns, 300 microns, 250 microns, or 100 microns, and wherein the average diameter may range from any lower limit to any upper limit and encompass any range therebetween.

In some embodiments, the porous network of a porous implant of the present invention may have a porosity of about 40% to about 90%. The dimensions of such pores may depend on, inter alia, the size of the polymeric particles, the size of the fusions, and any additives included in the porous implant.

In some embodiments, the porosity of the porous network may range from a lower limit of about 1%, 5%, 10%, 20%, 30%, 40%, 50%, or 60% to an upper limit of about 99%, 90%, 80%, 70%, or 60%, and wherein the porosity may range from any lower limit to any upper limit and encompass any range therebetween. A lower porosity may exist if the porous implant has been compacted.

Antioxidants may, in some embodiments, mitigate oxidation and/or chemical degradation of the polymeric particles described herein during storage, transportation, and/or implementation (in vivo and/or ex vivo). Examples of antioxidants suitable for use in conjunction with the porous implants of the present invention may, in some embodiments, include, but are not limited to, anthocyanin, ascorbic acid, glutathione, lipoic acid, uric acid, resveratrol, flavonoids, carotenes (e.g., beta-carotene), carotenoids, tocopherols (e.g., alpha-tocopherol, beta-tocopherol, gamma-tocopherol, and delta-tocopherol), tocotrienols, ubiquinol, melatonin, secondary aromatic amines, benzofuranones, hindered phenols, polyphenols, hindered amines, organophosphorus compounds, thioesters, benzoates, lactones, hydroxylamines, and the like, and any combination thereof.

In some embodiments, antioxidants may be included in the porous implants of the present invention in an amount ranging from a lower limit of about 0.001%, 0.01%, 0.05%, 0.1%, 0.5%, or 1% an upper limit of about 10%, 5%, 3%, 2%, or 1% by weight of the polymer, wherein the amount may range from any lower limit to any upper limit and encompass any subset therebetween. The amount of the antioxidants may depend on a number of factors that include, inter alia, the application of the porous implant, the composition of the antioxidant, the composition of the polymeric particles, and any additional ingredients utilized in conjunction with the porous implant (described further herein).

In some embodiments, porous implants of the present invention may further comprises additives like active pharmaceutical ingredients (“APIs”), growth factors, cells or cell-like structures, imaging agents, pigments and/or dyes, and the like, and any combination thereof. It should be noted that the term “API” encompasses prodrugs.

Porous implants of the present invention may, in some embodiments, comprise a plurality of polymeric particles sintered together at a plurality of contact points so as to form a porous network, at least one antioxidant, and optionally further comprise at least one API, wherein the antioxidant and the API are independently adsorbed to a surface of at least a portion of the polymeric particles and/or disposed in a pore of the porous network.

Nonlimiting examples of APIs suitable for use in conjunction with the porous implants of the present invention include, but are not limited to, 16-alpha fluoroestradiol, 16-alpha-gitoxin, 16-epiestriol, 17-alpha dihydroequilenin, 17-alpha estradiol, 17-beta estradiol, 17-hydroxy progesterone, 1-alpha-hydroxyvitamin D2, 1-dodecpyrrolidinone, 20-epi-1,25 dihydroxyvitamin D3, 22-oxacalcitriol, 2CW, 2′-nor-cGMP, 3-isobutyl GABA, 5-ethynyluracil, 6-FUDCA, 7-methoxytacrine, abamectin, abanoquil, abcizimab (commercially available as REOPRO® from Eli Lilly and Company), abecarnil, abiraterone, ablukast, ablukast sodium, acadesine, acamprosate, acarbose, acebutolol, acecainide hydrochloride, aceclidine, aceclofenae, acedapsone, aceglutamide aluminum, acemannan, acetaminophen, acetazolamide, acetohexamide, acetohydroxamic acid, acetomepregenol, acetophenazine maleate, acetosulfone sodium, acetylcholine chloride, acetylcysteine, acetyl-L-carnitine, acetylmethadol, acifran, acipimox, acitemate, acitretin, acivicin, aclarubicin, aclatonium, acodazole hydrochloride, aconiazide, acrisorcin, acrivastine, acronine, actisomide, actodigin, acyclovir, acylfulvene, adafenoxate, adalimumab (commercially available as HUMIRA® from Abbott Laboratories), adapalene, adapalene, adatanserin, adatanserin hydrochloride, adecypenol, adecypenol, adefovir, adelmidrol, ademetionine, adenosine, adinazolam, adipheinine hydrochloride, adiposin, adozelesin, adrafinil, adrenalone, airbutamine, alacepril, alamecin, alanine, alaproclate, alaptide, albendazole, albolabrin, albuterol (commercially available as VENTOLIN® from

GlaxoSmithKline), albutoin, alclofenae, alclometasone dipropionate, aluminum chlorhydroxyallantoinate (commercially available as ALCOLOXA® from TRI-K Industries, Inc.), aldecalmycin, aldesleukin, aldioxa, alendronate sodium (commercially available as FOSAMAX® from Merck), alendronic acid, alentemol, alentemol hydrobromide, aletamine hydrochloride, aleuronium chloride, alexidine, alfacalcidol, alfentanil hydrochloride, alfuzosin, algestone acetonide, alglucerase, aliflurane, alinastine, alipamide, allantoin, allobarbital, allopurinol, a tachy-kinins (TK) antagonist, alonimid, alosetron, alosetron hydrochloride, alovudine, alpertine, alpha amylase, alpha idosone, alpidem, alprazolam (commercially available as XANAX® from Pfizer, Inc.), alprenolol hydrochloride, alprenoxime hydrochloride, alprostadil, alrestatin sodium, altanserin tartrate, alteplase, althiazide, altretamine, altromycin B, alverinc citrate, alvircept sudotox, amadinone acetate, amantadine hydrochloride, ambamustine, ambomycin, ambruticin, ambuphylline, ambuside, amcinafal, amcinonide, amdinocillin, amdinocillin pivoxil, amedalin hydrochloride, amelometasone, ameltolide, amesergide, ametantrone acetate, amezinium metilsulfate, amfebutamone, amfenac sodium, amflutizole, amicycline, amidephrine mesylate, amidox, amifloxacin, amifostine, amikacin, amiloride hydrochloride, aminacrine hydrochloride, aminobenzoate potassium, aminobenzoate sodium, aminocaproic acid, aminoglutethimide, aminohippurate sodium, aminolevulinic acid, aminophylline, aminorex, aminosalicylate sodium, aminosalicylic acid, amiodarone, amiprilose hydrochloride, amiquinsin hydrochloride, amisulpride, amitraz, amitriptyline hydrochloride, amlexanox, amlodipine, amobarbital sodium, amodiaquine, amodiaquine hydrochloride, amorolfine, amoxapine, amoxicillin, amphecloral, amphetamine sulfate, amphomycin, amphotericin B, ampicillin, ampiroxicam, ampyzine sulfate, amquinate, amrinone, aminone, amrubicin, amsacrine, amythiamicin, anagestone acetate, anagrelide, anakinra, ananain, anaritide, anaritide acetate, anastrozole (commercially available as ARIMIDEX® from AstraZeneca), anazolene sodium, ancrod, andrographolide, androstenedione, angiogenesis inhibitors, angiotensin amide, anidoxime, anileridine, anilopam hydrochloride, aniracetam, anirolac, anisotropine methylbromide, anistreplase, anitrazafen, anordrin, antagonist D, antagonist G, antarelix, antazoline phosphate, anthelmycin, anthralin, anthramycin, antiandrogen, antihemophilic factor (commercially available as XYNTHA® from Pfizer, Inc.), acedapsone, felbamate, antiestrogen, antineoplaston, antipyrine, antisense oligonucleotides, apadoline, apafant, apalcillin sodium, apaxifylline, apazone, aphidicolin glycinate, apixifylline, apomorphine hydrochloride, apraclonidine, apraclonidine hydrochloride, apramycin, aprindine, aprindine hydrochloride, aprosulate sodium, aprotinin, aptazapine maleate, aptiganel, apurinic acid, apurinic acid, aranidipine, aranotin, arbaprostil, arbekicin, 1-methyl-2-((phenylthio) methyl)-3-carbethoxy-4-((dimethylamino) methyl)-5-hydroxy-6-bromindole (commercially available as ARBIDOL® from Masterlek), arbutamine hydrochloride, arclofenin, ardeparin sodium, (2R,4R)-1-[(2S)-5-(diaminomethylideneamino) -2-[[(3R)-3-methyl-1,2,3,4-tetrahydroquinolin-8-yl] sulfonylamino] pentanoyl]-4-methyl-piperidine-2-carboxylic acid (commercially available as ARGATROBAN® from GlaxoSmithKline), arginine, argipressin tannate, arildone, aripiprazol, arotinolol, arpinocid, arteflene, artilide fumarate, asimadoline, aspalatone, asparaginase, aspartic acid, aspartocin, asperfuran, aspirin, aspoxicillin, asprelin, astemizole, astromicin sulfate, asulacrine, atamestane, atenolol, atevirdine, atipamezole, atiprosin maleate, atolide, atorvastatin (commercially available as LIPITOR® from Pfizer, Inc.), atosiban, atovaquone, atpenin B, atracurium besylate, atrimustine, atrinositol, atropine, auranofin, aureobasidin A, aurothioglucose, avilamycin, avoparcin, avridine, nizatidine (commercially available as AXID® from GlaxoSmithKline), axinastatin 1, axinastatin 2, axinastatin 3, azabon, azacitidinie, azaclorzine hydrochloride, azaconazole, azadirachtine, azalanstat dihydrochloride, azaloxan fumarate, azanator maleate, azanidazole, azaperone, azaribine, azaserine, azasetron, azatadine maleate, azathioprine, azathioprine sodium, azatoxin, azatyrosine, azelaic acid, azelastine, azelnidipine, azepindole, azetepa, azimilide, azithromycin, azlocillin, azolimine, azosemide, azotomycin, aztreonam, azumolene sodium, bacampicillin hydrochloride, baccatin III, bacitracin, baclofen, bacoside A, bacoside B, bactobolamine, balanol, balazipone, balhimycin, balofloxacin, balsalazide, bambermycins, bambuterol, bamethan sulfate, bamifylline hydrochloride, bamidazole, baohuoside 1, barmastine, barnidipine, basifungin, batanopride hydrochloride, batebulast, batelapine maleate, batimastat, beauvericin, becanthone hydrochloride, becaplermin, becliconazole, beclomethasone dipropionate, befloxatone, beinserazide, belfosdil, belladonna, beloxamide, bemesetron, bemitradine, bemoradan, benapryzine hydrochloride, benazepril hydrochloride, benazeprilat, bendacalol mesylate, bendazac, bendroflumethiazide, benflumetol, benidipine, benorterone, benoxaprofen, benoxaprofen, benoxinate hydrochloride, benperidol, bentazepam, bentiromide, benurestat, benzbromarone, benzethonium chloride, benzetimide hydrochloride, benzilonium bromide, benzindopyrine hydrochloride, benzisoxazole, benzocaine, benzochlorins, benzoctamine hydrochloride, benzodepa, benzoidazoxan, benzonatate, benzoyl peroxide, benzoylpas calcium, benzoylstaurosporine, benzquinamide, benzthiazide, benztropine, benztropine mesylate, benzydamine hydrochloride, benzylpenicilloyl polylysine, bepridil, bepridil hydrochloride, beractant, beraprost, berefrine, berlafenone, bertosamil, berythromycin, besipirdine, beta-alethine, betaclamycin B, betamethasone, betamipron, betaxolol, betaxolol hydrochloride, bethanechol chloride, bethanidine sulfate, betulinic acid, bevacizumab (commercially available as AVASTIN® available from Genenetech), bevantolol, bevantolol hydrochloride, bezafibrate, bFGF inhibitor, bialamicol hydrochloride, biapenem, bicalutamide, bicifadine hydrochloride, biclodil hydrochloride, bidisomide, bifemelane, bifonazole, bimakalim, bimithil, bindarit, biniramycin, binospirone, bioxalomycin alpha2, bipenamol hydrochloride, biperiden, biphenamine hydrochloride, biriperone, bisantrene, bisaramil, bisaziridinylspermine, bis-benzimidazole A, bis-benzimidazole B, bisnafide, bisobrin lactate, bisoprolol, bispyrithione magsulfex, bistramide D, bistramide K, bistratene A, bithionolate sodium, bitolterol besylate, bivalirudin, bizelesin, bleomycin sulfate, bolandiol dipropionate, bolasterone, boldenone undecylenate, boldine, bolenol, bolmantalate, bopindolol, bosentan, boxidine, brefeldin, breflate, brequinar sodium, bretazenil, bretylium bosylate, brifentanil hydrochloride, brimonidine, brinolase, brocresine, brocrinat, brofoxine, bromadoline maleate, bromazepam, bromchlorenone, bromelains, bromfenac, brominidione, bromocriptine, bromodiphenhydramine hydrochloride, bromoxamide, bromperidol, bromperidol decanoate, brompheniramine baleate, broperamole, bropirimine, brotizolam, bucamide maleate, bucindolol, buclizine hydrochloride, bucromarone, budesonide (commercially available as RHINOCORT® and ENTOCORT® from AstraZeneca), budipine, budotitane, buformin, bumetanide, bunaprolast, bunazosin, bunolol hydrochloride, bupicomide, bupivacaine hydrochloride, buprenorphine hydrochloride, bupropion hydrochloride, buramate, buserelin acetate, buspirone hydrochloride, busulfan, butabarbital, butacetin, butaclamol hydrochloride, butalbital, butamben, butamirate citrate, butaperazine, butaprost, butedronate tetrasodium, butenafine, buterizine, buthionine sulfoximine, butikacin, butilfenin, butirosin sulfate, butixirate, butixocort propionate, butoconazole nitrate, butonate, butopamine, butoprozine hydrochloride, butorphanol, butoxamine hydrochloride, butriptyline hydrochloride, cactinomycin, cadexomer iodine, caffeine, calanolide A, calcifediol, calcipotriene, calcipotriol, calcitonin, calcitriol, calcium undecylenate, calphostin C, calusterone, cambendazole, camonagrel, camptothecin derivatives, canarypox IL-2, candesartan, candicidin, candoxatril, candoxatrilat, caniglibose, canrenoate potassium, canrenone, capecitabine, capobenate sodium, capobenic acid, capreomycin sulfate, capromab, capsaicin, captopril, capuride, caracemide, carbachol, carbadox, carbamazepine, carbamide peroxide, carbantel lauryl sulfate, carbaspirin calcium, carbazeran, carbazomycin C, carbenicillin potassium, carbenoxolone sodium, carbetimer, carbetocin, carbidopa, carbidopa-levodopa, carbinoxamine maleate, carbiphene hydrochloride, carbocloral, carbocysteine, carbol-fuchsin, carboplatin, carboprost, carbovir, carboxamide-amino-triazole, carboxyamidotriazole, carboxymethylated beta-1,3-glucan, carbuterol hydrochloride, CaRest M3, carfentanil citrate, carisoprodol, carmantadine, carmustine, CARN 700, camidazole, caroxazone, carperitide, carphenazine maleate, carprofen, carsatrin succinate, cartazolate, carteolol, carteolol hydrochloride, cartilage derived inhibitor, carubicin hydrochloride, carumonam sodium, carvedilol, carvotroline, carvotroline hydrochloride, carzelesin, casein kinase inhibitors (ICOS), castanospermine, caurumonam, cebaracetam, cecropin B, cedefingol, cefaclor, cefadroxil, cefamandole, cefaparole, cefatrizine, cefazaflur sodium, cefazolin, cefbuperazone, cefcapene pivoxil, cefdaloxime pentexil tosilate, cefdinir, cefditoren pivoxil, cefepime, cefetamet, cefetecol, cefixime, cefluprenam, cefinenoxime hydrochloride, cefinetazole, cefminlox, cefodizime, cefonicid sodium, cefoperazone sodium, ceforanide, cefoselis, cefotaxime sodium, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftibuten, ceftizoxime sodium, ceftriaxone, cefuroxime, celastrol, celikalim, celiprolol, cepacidiine A, cephacetrile sodium, cephalexin, cephaloglycin, cephaloridine, cephalothin sodium, cephapirin sodium, cephradine, cericlamine, cerivastatin, ceronapril, certoparin sodium, ceruletide, cetaben sodium, cetalkonium chloride, cetamolol hydrochloride, cetiedil, cetirizine, cetophenicol, cetraxate hydrochloride, cetrorelix, cetuximab (commercially available as ERBITUX® from Eli Lilly and Company), cetylpyridinium chloride, chenodiol, chlophedianol hydrochloride, chloral betaine, chlorambucil, chloramphenicol, chlordantoin, chlordiazepoxide, chlorhexidine gluconate, chlorins, chlormadinone acetate, chloroorienticin A, chloroprocaine hydrochloride, chloropropamide, chloroquine, chloroquinoxaline sulfonamide, chlorothiazide, chlorotrianisene, chloroxine, chloroxylenol, chlorphenesin carbamate, chlorpheniramine maleate, chlorpromazine, chlorpropamide, chlorprothixene, chlortetracycline bisulfate, chlorthalidone, chlorzoxazone, cholestyramine resin, chromonar hydrochloride, cibenzoline, cicaprost, ciclafrine hydrochloride, ciclazindol, ciclesonide, cicletanine, ciclopirox, cicloprofen, cicloprolol, cidofovir, cidoxepin hydrochloride, cifenline, ciglitazone, ciladopa hydrochloride, cilansetron, cilastatin sodium, cilazapril, cilnidipine, cilobamine mesylate, cilobradine, cilofungin, cilostazol, cimaterol, cimetidine, cimetropium bromide, cinalukast, cinanserin hydrochloride, cinepazet maleate, cinflumide, cingestol, cinitapride, cinnamedrine, cinnarizine, cinolazepam, cinoxacin, cinperene, cinromide, cintazone, cintriamide, cioteronel, cipamfylline, ciprefadol succinate, ciprocinonide, ciprofibrate, ciprofloxacin, ciprostene, ciramadol, cirolemycin, cisapride, cisatracurium besilate, cisconazole, cisplatin, cis-porphyrin, cistinexine, citalopram, citenamide, citicoline, citreamicin alpha, cladribine, clamoxyquin hydrochloride, clarithromycin, clausenamide, clavulanate potassium, clazolam, clazolimine, clebopride, clemastine, Clentiazem maleate, clidinium bromide, clinafloxacin, clindamycin, clioquinol, clioxamide, cliprofen, clobazam, clobetasol propionate, clobetasone butyrate, clocortolone acetate, clodanolene, clodazon hydrochloride, clodronic acid, clof azimine, clofibrate, clofilium phosphate, clogestone acetate, clomacran phosphate, clomegestone acetate, clometherone, clomethiazole, clomifene analogues, clominorex, clomiphene, clomipramine hydrochloride, clonazepam, clonidine, clonitrate, clonixeril, clonixin, clopamide, clopenthixol, cloperidone hydrochloride, clopidogrel (commercially available as PLAVIX® from Bristol-Myers Squibb and Sanofi Pharmaceuticals), clopimozide, clopipazan mesylate, clopirac, cloprednol, cloprostenol sodium, clorazepate dipotassium, clorethate, clorexolone, cloroperone hydrochloride, clorprenaline hydrochloride, clorsulon, clortermine hydrochloride, closantel, closiramine aceturate, clothiapine, clothixamide maleate cloticasone propionate, clotrimazole, cloxacillin benzathine, cloxyquin, clozapine, cocaine, coccidioidin, codeine, codoxime, colchicine, colestimide, colestipol hydrochloride, colestolone, colforsin, colfosceril palmitate, colistimethate sodium, colistin sulfate, collismycin A, collismycin B, colterol mesylate, combretastatin A4, combretastatin analogue, complestatin, conagenin, conorphone hydrochloride, contignasterol, contortrostatin, cormethasone acetate, corticorelin ovine triflutate, corticotropin, cortisone acetate, cortivazol, cortodoxone, cosalane, costatolide, cosyntropin, cotinine, warfarin (commercially available as COUMADIN® from Bristol-Myers Squibb), coumermycin, crambescidin 816, crilvastatin, crisnatol, cromitrile sodium, cromolyn sodium, crotamiton, cryptophycin 8, cucumariosid, cuprimyxin, curacin A, curdlan sulfate, zinc hyaluran (commercially available as CURIOSIN® from Gedeon

Richter), cyclacillin, cyclazocine, cyclazosin, cyclic HPMPC, cyclindole, cycliramine maleate, cyclizine, cyclobendazole, cyclobenzaprine, cyclobut A, cyclobut G, cyclocapron, cycloguanil pamoate, cycloheximide, cyclopentanthraquinones, cyclopenthiazide, cyclopentolate hydrochloride, cyclophenazine hydrochloride, cyclophosphamide, cycloplatam, cyclopropane, cycloserine, cyclosin, cyclosporine, cyclothialidine, cyclothiazide, cyclothiazomycin, cyheptamide, cypemycin, cypenamine hydrochloride, cyprazepam, cyproheptadine hydrochloride, cyprolidol hydrochloride, cyproterone, cyproximide, cysteamine, cysteine hydrochloride, cystine, cytarabine, cytarabine hydrochloride, cytarabine ocfosfate, cytochalasin B, cytolytic factor, cytostatin, dacarbazine, dacliximab, dactimicin, dactinomycin, daidzein, daledalin tosylate, dalfopristin, dalteparin sodium, daltroban, dalvastatin, danaparoid, danazol, dantrolene, daphlnodorin A, dapiprazole, dapitant, dapoxetine hydrochloride, dapsone, daptomycin, darglitazone sodium, darifenacin, darlucin A, darodipine, darsidomine, darusentan, daunorubicin hydrochloride, dazadrol maleate, dazepinil hydrochloride, dazmegrel, dazopride fumarate, dazoxiben hydrochloride, debrisoquin sulfate, decitabine, deferiprone, deflazacort, dehydrocholic acid, dehydrodidemnin B, dehydroepiandrosterone, delapril, delapril hydrochloride, delavirdine mesylate, delequamine, delfaprazine, delmadinone acetate, delmopinol, delphinidin, demecarium bromide, demeclocycline, demecycline, demoxepam, denofungin, deoxypyridinoline, 2-propylpentanoic acid (commercially available as DEPAKOTE® from Abbott), deprodone, deprostil, depsidomycin, deramciclane, dermatan sulfate, desciclovir, descinolone acetonide, desflurane, desipramine hydrochloride, desirudin, deslanoside, deslorelin, desmopressin, desogestrel, desonide, desoximetasone, desoxoamiodarone, desoxycorticosterone acetate, detajmium bitartrate, deterenol hydrochloride, detirelix acetate, devazepide, dexamethasone, dexamisole, dexbrompheniramine maleate, dexchlorpheniramine maleate, dexclamol hydrochloride, dexetimide, dexfenfluramine hydrochloride, dexifosfamide, deximafen, dexivacaine, dexketoprofen, dexloxiglumide, dexmedetomidine, dexormaplatin, dexoxadrol hydrochloride, dexpanthenol, dexpemedolac, dexpropranolol hydrochloride, dexrazoxane, dexsotalol, dextrin 2-sulphate, dextroamphetamine, dextromethorphan, dextrorphan hydrochloride, dextrothyroxine sodium, dexverapamil, dezaguanine, dezinamide, dezocine, diacetolol hydrochloride, diamocaine cyclamate, diapamide, diatrizoate meglumine, diatrizoic acid, diaveridine, diazepam, diaziquone, diazoxide, dibenzepin hydrochloride, dibenzothiophene, dibucaine, dichliorvos, dichloralphenazone, dichlorphenamide, dicirenone, diclofenac sodium, dicloxacillin, dicranin, dicumarol, dicyclomine hydrochloride, didanosine, didemnin B, didox, dienestrol, dienogest, diethylcarbamazine citrate, diethylhomospermine, diethylnorspermine, diethylpropion hydrochloride, diethylstilbestrol, difenoximide hydrochloride, difenoxin, diflorasone diacetate, difloxacin hydrochloride, difluanine hydrochloride, diflucortolone, diflumidone sodium, diflunisal, difluprednate, diftalone, digitalis, digitoxin, digoxin, dihexyverine hydrochloride, dihydrexidine, dihydro-5-azacytidine, dihydrocodeine bitartrate, dihydroergotamine mesylate, hihydroestosterone, dihydrostreptomycin sulfate, dihydrotachysterol, dihydrotaxol, phenytoin (commercially available as DILANTIN® from Parke, Davis & Company), dilevalol hydrochloride, diltiazem hydrochloride, dimefadane, dimefline hydrochloride, dimenhydrinate, dimercaprol, dimethadione, dimethindene maleate, dimethisterone, dimethyl prostaglandin A1, dimethyl sulfoxide, dimethylhomospermine, dimiracetam, dimoxamine hydrochloride, dinoprost, dinoprostone, dioxadrol hydrochloride, dioxamycin, diphenhydramine citrate, diphenidol, diphenoxylate hydrochloride, diphenyl spiromustine, dipivefin hydrochloride, dipivefrin, dipliencyprone, diprafenone, dipropylnorspermine, dipyridamole, dipyrithione, dipyrone, dirithromycin, discodermolide, disobutamide, disofenin, disopyramide, disoxaril, disulfuram, ditekiren, divalproex sodium, dizocilpine maleate, dobutamine, docarpamine, docebenone, docetaxel, doconazole, docosanol, dofetilide, dolasetron, drotrecogin alfa (commercially available as XIGRIS® from Eli Lilly and Company), duloxetine hydrochloride (commercially available as CYMBALTA® from Eli Lilly and Company), ebastine, ebiratide, ebrotidine, ebselen, ecabapide, ecabet, ecadotril, ecdisteron, echicetin, echistatin, echothiophate iodide, eclanamine maleate, eclazolast, ecomustine, econazole, ecteinascidin 722, edaravone, edatrexate, edelfosine, edifolone acetate, edobacomab, edoxudine, edrecolomab, edrophonium chloride, edroxyprogesteone acetate, efegatran, eflornithine, efonidipine, egualcen, elantrine, eleatonin, elemene, eletriptan, elgodipine, eliprodil, elsamitrucin, eltenae, elucaine, emalkalim, emedastine, emetine hydrochloride, emiglitate, emilium tosylate, emitefur, emoctakin, enadoline hydrochloride, enalapril, enalaprilat, enalkiren, enazadrem, encyprate, endralazine mesylate, endrysone, enflurane, englitazone, enilconazole, enisoprost, enlimomab, enloplatin, enofelast, enolicam sodium, enoxacin, enoxacin, enoxaparin sodium, enoxaparin sodium, enoximone, enpiroline phosphate, enprofylline, enpromate, entacapone, enterostatin, enviradene, enviroxime, ephedrine, epicillin, epimestrol, epinephrine, epinephryl borate, epipropidine, epirizole, epirubicin, epitetracycline hydrochloride, epithiazide, epoetin alfa, epoetin beta, epoprostenol, epoprostenol sodium, epoxymexrenone, epristeride, eprosartan, eptastigmine, equilenin, equilin, erbulozole, erdosteine, ergoloid mesylates, ergonovine maleate, ergotamine tartrate, ersentilide, ersofermin, erythritol, erythrityl tetranitrate, erythromycin, esmolol hydrochloride, esomeprazole (commercially available as NEXIUM® from AstraZeneca), esorubicin hydrochloride, esproquin hydrochloride, estazolam, estradiol, estramustine, estramustine analogue, estrazinol hydrobromide, estriol, estrofurate, estrogen agonists, estrogen antagonists, estrogens, conjugated estrogens, esterified, estrone, estropipate, esuprone, etafedrine hydrochloride, etanidazole, etanterol, etarotene, etazolate hydrochloride, eterobarb, ethacizin, ethacrynate sodium, ethacrynic acid, ethambutol hydrochloride, ethamivan, ethanolamine oleate, ethehlorvynol, ether, ethinyl estradiol, ethiodized oil, ethionamide, ethonam nitrate, ethopropazine hydrochloride, ethosuximide, ethotoin, ethoxazene hydrochloride, ethybenztropine, ethyl chloride, ethyl dibunate, ethylestrenol, ethyndiol, ethynerone, ethynodiol diacetate, etibendazole, etidocaine, etidronate disodium, etidronic acid, etifenin, etintidine hydrochloride, etizolam, etodolac, etofenamate, etoformin hydrochloride, etomidate, etonogestrel, etoperidone hydrochloride, etoposide, etoprine, etoxadrol hydrochloride, etozolin, etrabamine, etretinate, etryptamine acetate, eucatropine hydrochloride, eugenol, euprocin hydrochloride, eveminomicin, exametazine, examorelin, exaprolol hydrochloride, exemestane, exetimibe (commercially available as ZETIA® from Merck), fadrozole, faeriefungin, famciclovir, famotidine (commercially available as PEPCID® from Merck), fampridine, fantof arone, fantridone hydrochloride, faropenem, fasidotril, fasudil, fazarabine, fedotozine, felbamate, felbinac, felodipine, felypressin, fenalamide, fenamole, fenbendazole, fenbufen, fencibutirol, fenclofenac, fenclonine, fenclorac, fendosal, fenestrel, fenethylline hydrochloride, fenfluramine hydrochloride, fengabine, fenimide, fenisorex, fenmetozole hydrochloride, fenmetramide, fenobam, fenoctimine sulfate, fenofibrate, fenoldopam, fenoprofen, fenoterol, fenpipalone, fenprinast hydrochloride, fenprostalene, fenquizone, fenretinide, fenspiride, fentanyl citrate, fentiazac, fenticlor, fenticonazole, fenyripol hydrochloride, fepradinol, ferpifosate sodium, ferristene, ferrixan, ferrous sulfate, ferumoxides, ferumoxsil, fetoxylate hydrochloride, fexofenadine, fezolamine fumarate, fiacitabine, fialuridine, fibrinogen I 125, filgrastim, filipin, finasteride (commercially available as PROPECIA® from Merck), flavodilol maleate, flavopiridol, flavoxate hydrochloride, flazalone, flecainide, flerobuterol, fleroxacin, flesinoxan, flestolol sulfate, fletazepam, flezelastine, flobufen, floctafenine, flomoxef, flordipine, florfenicol, florifenine, flosatidil, flosequinan, floxacillin, floxuridine, fluasterone, fluazacort, flubanilate hydrochloride, flubendazole, flucindole, flucloronide, fluconazole, flucytosine, fludalanine, fludarabine phosphate, fludazonium chloride, fludeoxyglucose F 18, fludorex, fludrocortisone acetate, flufenamic acid, flufenisal, flumazenil, flumecinol, flumequine, flumeridone, flumethasone, flumetramide, flumezapine, fluminorex, flumizole, flumoxonide, flunarizine, flunidazole, flunisolide, flunitrazepam, flunixin, fluocalcitriol, fluocinolone acetonide, fluocinonide, fluocortin butyl, fluocortolone, fluorescein, fluorodaunorunicin hydrochloride, fluorodopa F 18, fluoroformylone, fluoroquinolones, fluorometholone, fluorouracil, fluotracen hydrochloride, fluoxetine, fluoxymesterone, fluparoxan, fluperamide, fluperolone acetate, fluphenazine decanoate, flupirtine, fluprednisolone, fluproquazone, fluprostenol sodium, fluquazone, fluradoline hydrochloride, flurandrenolide, flurazepam hydrochloride, flurbiprofen, fluretofen, flurithromycin, fluorocitabine, fluorof amide, fluorogestone acetate, flurothyl, fluoroxene, fluspiperone, fluspirilene, fluticasone propionate (commercially available as ADVAIR® from GlaxoSmithKline), fluticasone furoate, flutrimazole, flutroline, fluvastatin, fluvastatin sodium, fluvoxamine, fluzinamide, folic acid, follicle regulatory protein, folliculostatin, fomepizole, fonazine mesylate, forasartan, forfenimex, forfenirmex, formestane, formocortal, formoterol, fosarilate, fosazepam, foscarnet sodium, fosfomycin, fosfonet sodium, fosinopril, fosinoprilat, fosphenyloin, fosquidone, fostedil, fostriecin, fotemustine, fuchsin, basic, fumoxicillin, fungimycin, furaprofen, furazolidone, furazolium chloride, furegrelate sodium, furobufen, furodazole, furosemide, fusidate sodium, fusidic acid, gabapentin, gadobenate dimeglumine, gadobenic acid, gadobutrol, gadodiamide, gadolinium texaphyrin, gadopentetate dimegiumine, gadoteric acid, gadoteridol, gadoversetamide, galantamine, galdansetron, galdansetron hydrochloride, gallamine triethiodide, gallium nitrate, gallopamil, galocitabine, gamfexine, gamolenic acid, ganciclovir, ganirelix, ganirelix acetate, gelatinase inhibitors, gemcadiol, gemcitabine (commercially available as GEMZAR® from Eli Lilly and Company), gemeprost, gemfibrozil, gentamicin sulfate, gentian violet, gepirone, gestaclone, gestodene, gestonorone caproate, gestrinone, gevotroline hydrochloride, girisopam, glaspimod, glaucocalyxin A, glemanserin, gliamilide, glibornuride, glicetanile sodium, gliflumide, glimepiride, glipizide, gloximonam, glucagon, glutapyrone, glutathione inhibitors, glutethimide, glyburide, glycopine, glycopril, glycopyrrolate, glyhexamide, glymidine sodium, glyoctamide, glyparamide, colloidal gold Au 198, gonadoctrinins, gonadorelin, gonadotropins, goserelin, gramicidin, granisetron, grepafloxacin, griseofulvin, guaiapate, guaithylline, guanabenz, guanabenz acetate, guanadrel sulfate, guancydine, guanethidine monosulfate, guanfacine hydrochloride, guanisoquin sulfate, guanoclor sulfate, guanoctine hydrochloride, guanoxabenz, guanoxan sulfate, guanoxyfen sulfate, gusperimus trihydrochloride, halazepam, halcinonide, halichondrin B, halobetasol propionate, halof antrine, halof antrine hydrochloride, halofenate, halofuginone hydrobromide, halomon, galopemide, galoperidol, halopredone, haloprogesterone, haloprogin, halothane, halquinols, hamycin, han menopausal gonadotropins, hatomamicin, hatomarubigin A, hatomarubigin B, hatomarubigin C, hatomarubigin D, heparin sodium, hepsulfam, heregulin, hetacillin, heteronium bromide, hexachlorophene: hydrogen peroxide, hexafluorenium bromide, hexamethylene bisacetamide, hexedine, hexobendine, hexoprenaline sulfate, hexylresorcinol, histamine phosphate, histidine, histoplasmin, histrelin, homatropine hydrobromide, hoquizil hydrochloride, human chorionic gonadotropin, hycanthone, hydralazine hydrochloride, hydralazine polistirex, hydrochlorothiazide, hydrocodone bitartrate, hydrocortisone, hydroflumethiazide, hydromorphone hydrochloride, hydroxyamphetamine hydrobromide, hydroxychloroquine sulfate, hydroxyphenamate, hydroxyprogesterone caproate, hydroxyurca, hydroxyzine hydrochloride, hymecromone, hyoscyamine, hypericin, ibafloxacin, ibandronic acid, ibogaine, ibopamine, ibudilast, ibufenac, ibuprofen, ibutilide fumarate, icatibant acetate, ichthammol, icotidine, idarubicin, idoxifene, idoxuridine, idramantone, iemefloxacin, iesopitron, ifetroban, ifosfamide, ilepeimide, illimaquinone, ilmofosine, ilomastat, ilonidap, iloperidone, iloprost, imafen hydrochloride, imazodan hydrochloride, imidapril, imidazenil, imidazoacridones, imidecyl iodine, imidocarb hydrochloride, imidoline hydrochloride, imidurea, imiloxan hydrochloride, imipenem, imipramine hydrochloride, imiquimod, immunostimulant peptides, impromidine hydrochloride, indacrinone, indapamide, indecainide hydrochloride, indeloxazine hydrochloride, indigotindisulfonate sodium, indinavir, indocyanine green, indolapril hydrochloride, indolidan, indometacin, indomethacin sodium, indoprofen, indoramin, indorenate hydrochloride, indoxole, indriline hydrochloride, infliximab (commercially available as REMICADE® from Janssen Biotech, Inc.), inocoterone, inogatran, inolimomab, inositol niacinate, insulin, insulin glargine (commercially available as LANTUS® from Sanofi-Aventis), interferons, interferon beta-1a (commercially available as AVONEX® from BIOGEN), interleukins, intrazole, intriptyline hydrochloride, iobenguane, iobenzamic acid, iobitridol, iocarmate meglumine, iocarmic acid, iocetamic acid, iodamide, iodine, iodipamide meglumine, iodixanol, iodoamiloride, iodoantipyrine I 131, iodocholesterol I 131, iododoxorubicin, iodohippurate sodium I 131, iodopyracet I 125, iodoquinol, iodoxamate meglumine, iodoxamie acid, ioglicic acid, iofetamine hydrochloride I 123, iofratol, ioglucol, ioglucomide, ioglycamic acid, iogulamide, iohexyl, iomeprol, iomethin I 125, iopamidol, iopanoic acid, iopentol, iophendylate, ioprocemic acid, iopromide, iopronic acid, iopydol, iopydone, iopyrol, iosefamic acid, ioseric acid, iosulamide meglumine, iosumetic acid, iotasul, iotetric acid, iothalamate sodium, iothalamic acid, iotriside, iotrolan, iotroxic acid, iotyrosine I 131, ioversol, ioxagiate sodium, ioxaglate meglumine, ioxaglic acid, ioxilan, ioxotrizoic acid, ipazilide, ipenoxazone, ipidacrine, ipodate calcium, ipomeanol, 4-, ipratropium bromide, ipriflavone, iprindole, iprofenin, ipronidazole, iproplatin, iproxamine hydrochloride, ipsapirone, irbesartan, irinotecan, irloxacin, iroplact, irsogladine, irtemazole, isalsteine, isamoxole, isbogrel, isepamicin, isobengazole, isobutamben, isocarboxazid, isoconazole, isoetharine, isofloxythepin, isoflupredone acetate, isoflurane, isofluorophate, isohomohalicondrin B, isoleucine, isomazole hydrochloride, isomylamine hydrochloride, isoniazid, isopropamide iodide, isopropyl alcohol, isopropyl unoprostone, isoproterenol hydrochloride, isosorbide, isosorbide mononitrate, isotiquimide, isotretinoin, isoxepac, isoxicam, isoxsuprine hydrochloride, isradipine, itameline, itasetron, itazigrel, itopride, itraconazole, ivermectin, jasplakinolide, josamycin, kahalalide F, kalafungin, kanamycin sulfate, ketamine hydrochloride, ketanserin, ketazocine, ketazolam, kethoxal, ketipramine fumarate, ketoconazole, ketoprofen, ketorfanol, ketorolac, ketotifen fumarate, kitasamycin, labetalol hydrochloride, lacidipine, lacidipine, lactitol, lactivicin, laennec, lafutidine, lamellarin-n triacetate, lamifiban, lamivudine, lamotrigine, lanoconazole, LANOXIN® (digoxin, available from GlaxoSmithKline), lanperisone, lanreotide, lansoprazole (commercially available as PREVAID® from Takeda Pharmaceuticals, Inc.), latanoprost, lateritin, laurocapram, lauryl isoquinolinium bromide, lavoltidine succinate, lazabemide, lecimibide, leinamycin, lemildipine, leminoprazole, lenercept, leniquinsin, lenograstim, lenperone, lentinan sulfate, leptin, leptolstatin, lercanidipine, lergotrile, lerisetron, letimide hydrochloride, letrazuril, letrozole, leucine, leucomyzin, leuprolide acetate, leuprolide, leuprorelin, levamfetamine succinate, levamisole, levdobutamine lactobionate, levcromakalim, levetiracetam, levobetaxolol, levobunolol, levobupivacaine, levocabastine, levocarnitine, levodopa, levodropropizine, levofloxacin (commercially available as LEVAQUIN® from Jessen Pharmaceuticals, Inc.), levofuraltadone, levoleucovorin calcium, levomethadyl acetate, levomethadyl acetate hydrochloride, levomoprolol, levonantradol hydrochloride, levonordefrin, levonorgestrel, levopropoxyphene napsylate, levopropylcillin potassium, levormeloxifene, levorphanol tartrate, levosimendan, levosulpiride, levothyroxine sodium, levoxadrol hydrochloride, lexipafant, lexithromycin, liarozole, libenzapril, lidamidine hydrochloride, lidocaine, lidofenin, lidoflazine, lifarizine, lifibrate, lifibrol, linarotene, lincomycin, linear polyamine analogue, linogliride, linopirdine, linotroban, linsidomine, lintitript, lintopride, liothyronine I 125, liothyronine sodium, liotrix, lirexapride, lisinopril, lissoclinamide 7, lixazinone sulfate, lobaplatin, lobenzarit sodium, lobucavir, lodelaben, lodoxamide, lofemizole hydrochloride, lofentanil oxalate, lofepramine hydrochloride, lofexidine hydrochloride, lombricine, lomefloxacin, lomerizine, lometraline hydrochloride, lometrexol, lomitapide, lomofungin, lornoxicam, lomustine, lonapalene, lonazolac, lonidamine, loperamide hydrochloride, loracarbef, lorajmine hydrochloride, loratadine, lorazepam, lorbamate, lorcainide hydrochloride, loreclezole, lorglumide, lormetazepam, lornoxicam, lornoxicam, lortalamine, lorzafone, losartan (commercially available as COZAAR® from Merck), losigamone, losoxantrone, losulazine hydrochloride, loteprednol, lovastatin, loviride, loxapine, loxoribine, lubeluzole, lucanthone hydrochloride, lufironil, lurosetron mesylate, lurtotecan, luteinizing hormone, lutetium, lutrelin acetate, luzindole, lyapolate sodium, lycetamine, lydicamycin, lydimycin, lynestrenol, lypressin, lysine, lysofylline, lysostaphin, lytic peptides, maduramicin, mafenide, magainin 2 amide, magnesium salicylate, magnesium sulfate, magnolol, maitansine, malethamer, mallotochromene, mallotojaponin, malotilate, mangafodipir, manidipine, maniwamycin A, mannitol, mannostatin A, manumycin E, manumycin F, MAPK/ERK kinase (MEK) inhibitors, mapinastine, maprotiline, marimastat, masoprocol, maspin, massetolide, matrilysin inhibitors, maytansine, mazapertine succiniate, mazindol, mebendazole, mebeverine hydrochloride, mebrofenin, mebutamate, mecamylamine hydrochloride, mechlorethamine hydrochloride, meclocycline, meclofenamate sodium, mecloqualone, meclorisone dibutyrate, medazepam hydrochloride, medorinone, medrogestone, medroxalol, medroxyprogesterone (commercially available as DEPO-PROVERA® from Pfizer, Inc.), medrysone, meelizine hydrochloride, mefenamic acid, mefenidil, mefenorex hydrochloride, mefexamide, mefloquine hydrochloride, mefruside, megalomicin potassium phosphate, megestrol acetate, meglumine, meglutol, melengestrol acetate, melitracen hydrochloride, melphalan, memotine hydrochloride, menabitan hydrochloride, menoctone, menogaril, menotropins, meobentine sulfate, mepartricin, mepenzolate bromide, meperidine hydrochloride, mephentermine sulfate, mephenyloin, mephobarbital, mepivacaine hydrochloride, meprobamate, meptazinol hydrochloride, mequidox, meralein sodium, merbarone, mercaptopurine, mercufenol chloride, mercury, meropenem, mesalamine, meseclazone, mesoridazine, mesterolone, mestranol, mesuprine hydrochloride, metalol hydrochloride, metaproterenol polistirex, metaraminol bitartrate, metaxalone, meteneprost, meterelin, metformin, methacholine chloride, methacycline, methadone hydrochloride, methadyl acetate, methalthiazide, methamphetamine hydrochloride, methaqualone, methazolamide, methdilazine, methenamine, methenolone acetate, methetoin, methicillin sodium, methimazole, methioninase, methionine, methisazone, methixene hydrochloride, methocarbamol, methohexital sodium, methopholine, methotrexate, methotrimeprazine, methoxatone, methoxyflurane, methsuximide, methyclothiazide, methyl 10 palmoxirate, methylatropine nitrate, methylbenzethonium chloride, methyldopa, methyldopate hydrochloride, methylene blue, methylergonovine maleate, methylhistamine, R-alpha, methylinosine monophosphate, methylphenidate hydrochloride, methylprednisolone, methyltestosterone, methynodiol diacelate, methysergide, methysergide maleate, metiamide, metiapine, metioprim, metipamide, metipranolol, metizoline hydrochloride, metkephamid acetate, metoclopramide, metocurine iodide, metogest, metolazone, metopimazine, metoprine, metoprolol, metoquizine, metrifonate, metrizamide, metrizoate sodium, metronidazole, meturedepa, metyrapone, metyrosine, mexiletine hydrochloride, mexrenoate potassium, mezlocillin, mfonelic acid, mianserin hydrochloride, mibefradil, mibefradil dihydrochloride, mibolerone, michellamine B, miconazole, microcolin A, midaflur, midazolam hydrochloride, midodrine, mifepristone, mifobate, miglitol, milacemide, milameline, mildronate, milenperone, milipertine, milnacipran, milrinone, miltefosine, mimbane hydrochloride, minaprine, minaxolone, minocromil, minocycline, minoxidil, mioflazine hydrochloride, miokamycin, mipragoside, mirfentanil, mirimostim, mirincamycin hydrochloride, mirisetron maleate, mirtazapine, mismatched double stranded RNA, misonidazole, misoprostol, mitindomide, mitocarcin, mitocromin, mitogillin, mitoguazone, mitolactol, mitomalcin, mitomycin, mitonafide, mitosper, mitotane, mitoxantrone, mivacurium chloride, mivazerol, mixanpril, mixidine, mizolastine, mizoribine, moclobemide, modafinil, modaline sulfate, modecamide, moexipril, mof arotene, mofegiline hydrochloride, mofezolac, molgramostim, molinazone, molindone hydrochloride, molsidomine, mometasone, monatepil maleate, monensin, monoctanoin, montelukast sodium (commercially available as SINGULAIR® available from Merck), montirelin, mopidamol, moracizine, morantel tartrate, moricizine, morniflumate, morphine, morphine sulfate, morrhuate sodium, mosapramine, mosapride, motilide, motretinide, moxalactam disodium, moxazocine, moxiraprine, moxnidazole, moxonidine, mumps skin test antigen, mustard anticancer agent, muzolimine, mycaperoxide B, mycophenolic acid, myriaporone, nabazenil, nabilone, nabitan hydrochloride, naboctate hydrochloride, nabumetone, n-acetyldinaline, nadide, nadifloxacin, nadolol, nadroparin calcium, nafadotride, nafamostat, nafarelin, nafcillin sodium, nafenopin, nafimidone hydrochloride, naflocort, nafomine malate, nafoxidine hydrochloride, nafronyl oxalate, naftifine hydrochloride, naftopidil, naglivan, nagrestip, nalbuphine hydrochloride, nalidixate sodium, nalidixic acid, nalmefene, nalmexone hydrochloride, naloxone/pentazocine, naltrexone, namoxyrate, nandrolone phenpropionate, nantradol hydrochloride, napactadine hydrochloride, napadisilate, napamezole hydrochloride, napaviin, naphazoline hydrochloride, naphterpin, naproxen, naproxol, napsagatran, naranol hydrochloride, narasin, naratriptan, nartograstim, nasaruplase, natamycin, nateplase, naxagolide hydrochloride, nebivolol, nebramycin, nedaplatin, nedocromil, nefazodone hydrochloride, neflumozide hydrochloride, nefopam hydrochloride, nelezaprine maleate, nemazoline hydrochloride, nemorubicin, neomycin palmitate, neostigmine bromide, neridronic acid, netilmicin sulfate, neutral endopeptidase, neutramycin, nevirapine, nexeridine hydrochloride, niacin, nibroxane, nicardipine hydrochloride, nicergoline, niclosamide, nicorandil, nicotinyl alcohol, nicotine (commercially available as NICOTROL® NS from Pfizer, Inc.), nifedipine, nifirmerone, nifluridide, nifuradene, nifuraldezone, nifuratel, nifuratrone, nifurdazil, nifurimide, nifurpirinol, nifurquinazol, nifurthiazole, nilutamide, nilvadipine, nimazone, nimodipine, niperotidine, niravoline, niridazole, nisamycin, nisbuterol mesylate, nisin, nisobamate, nisoldipine, nisoxetine, nisterime acetate, nitarsone, nitazoxamide, nitecapone, nitrafudam hydrochloride, nitralamine hydrochloride, nitramisole hydrochloride, nitrazepam, nitrendipine, nitrocycline, nitrodan, nitrofurantoin, nitrofurazone, nitroglycerin, nitromersol, nitromide, nitromifene citrate, nitrous oxide, nitroxide antioxidant, nitrullyn, nivazol, nivimedone sodium, nizatidine, noberastine, nocodazole, nogalamycin, nolinium bromide, nomifensine maleate, noracymethadol hydrochloride, norbolethone, norepinephrine bitartrate, norethindrone, norethynodrel, norfloxacin, norflurane, norgestimate, norgestomet, norgestrel, nortriptyline hydrochloride, noscapine, novobiocin sodium, N-substituted benzaimides, nufenoxole, nylestriol, nystatin, O6-benzylguanine, obidoxime chloride, ocaperidone, ocfentanil hydrochloride, ocinaplon, octanoic acid, octazamide, octenidine hydrochloride, octodrine, octreotide, octriptyline phosphate, ofloxacin, oformine, okicenone, olanzapine (commercially available as ZYPREXA® from Eli Lilly and Company), oligonucleotides, olopatadine, olprinone, olsalazine, olsalazine sodium, olvanil, omeprazole, onapristone, ondansetron, ontazolast, oocyte maturation inhibitor, opipramol hydrochloride, oracin, orconazole nitrate, orgotein, orlislat, ormaplatin, ormetoprim, ornidazole, orpanoxin, orphenadrine citrate, osaterone, otenzepad, oxacillin sodium, oxagrelate, oxaliplatin, oxamarin hydrochloride, oxamisole, oxamniquine, oxandrolone, oxantel pamoate, oxaprotiline hydrochloride, oxaprozin, oxarbazole, oxatomide, oxaunomycin, oxazepam, oxcarbazepine, oxendolone, oxethazaine, oxetorone fumarate, oxfendazole, oxfenicine, oxibendazole, oxiconazole, oxidopamine, oxidronic acid, oxifungin hydrochloride, oxilorphan, oximonam, oximonam sodium, oxiperomide, oxiracetam, oxiramide, oxisuran, oxmetidine hydrochloride, oxodipine, oxogestone phenpropionate, oxolinic acid, oxprenolol hydrochloride, oxtriphylline, oxybutynin chloride, oxychlorosene, oxycodone, oxymetazoline hydrochloride, oxymetholone, oxymorphone hydrochloride, oxypertine, oxyphenbutazone, oxypurinol, oxytetracycline, oxytocin, ozagrel, ozolinone, paclitaxel, palauamine, paldimycin, palinavir, paliperidone (commercially available as INVEGA® from Janssen Pharmaceuticals, Inc.), paliperidone palmitate (commercially available as INVEGA® SUSTENNA® from Janssen Pharmaceuticals, Inc.), palmitoylrhizoxin, palmoxirate sodium, pamaqueside, pamatolol sulfate, pamicogrel, pamidronate disodium, pamidronic acid, panadiplon, panamesine, panaxytriol, pancopride, pancuronium bromide, panipenem, pannorin, panomifene, pantethine, pantoprazole, papaverine hydrochloride, parabactin, parachlorophenol, paraldehyde, paramethasone acetate, paranyline hydrochloride, parapenzolate bromide, pararosaniline pamoate, parbendazole, parconazole hydrochloride, paregoric, pareptide sulfate, pargyline hydrochloride, parnaparin sodium, paromomycin sulfate, paroxetine (commercially available as PAXIL® from GlaxoSmithKlein), parthenolide, partricin, paulomycin, pazelliptine, pazinaclone, pazoxide, pazufloxacin, pefloxacin, pegaspargase, pegorgotein, pelanserin hydrochloride, peldesine, peliomycin, pelretin, pelrinone hydrochloride, pemedolac, pemerid nitrate, pemetrexed, pemirolast, pemoline, penamecillin, penbutolol sulfate, penciclovir, penfluridol, penicillin G benzathine, penicillin G potassium, penicillin G procaine, penicillin G Sodium, penicillin V, penicillin V benzathine, penicillin V hydrabamine, penicillin V potassium, pentabamate, pentaerythritol tetranitrate, pentafuside, pentamidine, pentamorphone, bentamustine, pentapiperium methylsulfate, pentazocine, pentetic acid, pentiapine maleate, pentigetide, pentisomicin, pentizidone sodium, pentobarbital, pentomone, pentopril, pentosan, pentostatin, pentoxifylline, pentrinitrol, pentrozole, peplomycin sulfate, pepstatin, perflubron, perfof amide, perfosfamide, pergolide, perhexyline maleate, perillyl alcohol, perindopril, perindoprilat, perlapine, permethrin, perospirone, perphenazine, phenacemide, phenaridine, phenazinomycin, phenazopyridine hydrochloride, phenbutazone sodium glycerate, phencarbamide, phencyclidine hydrochloride, phendimetrazine tartrate, phenelzine sulfate, phenmetrazine hydrochloride, phenobarbital, phenoxybenzamine hydrochloride, phenprocoumon, phenserine, phensuccinal, phensuximide, phentermine, phentermine hydrochloride, phentolamine mesilate, phentoxifylline, phenyl aminosalicylate, phenylacetate, phenylalanine, phenylalanyl ketoconazole, phenylbutazone, phenylephrine hydrochloride, phenylpropanolamine hydrochloride, phenylpropanolamine polistirex, phenyramidol hydrochloride, phenyloin, phosphatase inhibitors, physostigmine, picenadol, picibanil, picotrin diolamine, picroliv, picumeterol, pidotimod, pifamine, pilocarpine, pilsicamide, pimagedine, pimetine hydrochloride, pimilprost, pimobendan, pimozide, pinacidil, pinadoline, pindolol, pinnenol, pinocebrin, pinoxepin hydrochloride, pioglitazone (commercially available as ACTOS® from Takeda Pharmaceuticals), pipamperone, pipazethate, pipecuronium bromide, piperacetazine, piperacillin sodium, piperamide maleate, piperazine, pipobroman, piposulfan, pipotiazine palmitate, pipoxolan hydrochloride, piprozolin, piquindone hydrochloride, piquizil hydrochloride, piracetam, pirandamine hydrochloride, pirarubicin, pirazmonam sodium, pirazolac, pirbenicillin sodium, pirbuterol acetate, pirenperone, pirenzepine hydrochloride, piretanide, pirfenidone, piridicillin sodium, piridronate sodium, piriprost, piritrexim, pirlimycin hydrochloride, pirlindole, pirmagrel, pirmenol hydrochloride, pirnabine, piroctone, pirodavir, pirodomast, pirogliride tartrate, pirolate, pirolazamide, piroxantrone hydrochloride, piroxicam, piroximone, pirprofen, pirquinozol, pirsidomine, prenylamine, pitavastatin (commercially available as LIVALOA® from

Eli Lilly and Company), pituitary, posterior, pivampicillin hydrochloride, pivopril, pizotyline, placetin A, platinum compounds, platinum-triamine complex, plicamycin, plomestane, pobilukast edamine, podofilox, poisonoak extract, poldine methylsulfate, poliglusam, polignate sodium, polymyxin B sulfate, polythiazide, ponalrestat, porfimer sodium, porfiromycin, potassium chloride, potassium iodide, potassium permanganate, povidone-iodine, practolol, pralidoxime chloride, pramiracetam hydrochloride, pramoxine hydrochloride, pranolium chloride, prasugrel (commercially available as EFFIENT® from Eli Lilly and Company), pravadoline maleate, pravastatin, prazepam, prazosin, prazosin hydrochloride, prednazate, prednicarbate, prednimustine, prednisolone, prednisone, prednival, pregabalin (commercially available as LYRICA® from Pfizer, Inc.), pregnenolone succiniate, prenalterol hydrochloride, pridefine hydrochloride, prifelone, prilocalne hydrochloride, prilosec, primaquine phosphate, primidolol, primidone, prinivil, prinomide tromethamine, prinoxodan, prizidilol hydrochloride, proadifen hydrochloride, probenecid, probicromil calcium, probucol, procainamide hydrochloride, procaine hydrochloride, procarbazine hydrochloride, procaterol hydrochloride, prochlorperazine, procinonide, proclonol, procyclidine hydrochloride, prodilidine hydrochloride, prodolic acid, prof adol hydrochloride, progabide, progesterone, proglumide, proinsulin human, proline, prolintane hydrochloride, promazine hydrochloride, promethazine hydrochloride, propafenone hydrochloride, propagermanium, propanidid, propantheline bromide, proparacaine hydrochloride, propatyl nitrate, propentofylline, propenzolate hydrochloride, propikacin, propiomazine, propionic acid, propionylcarnitine, propiram, propiram+paracetamol, propiverine, propofol, propoxycaine hydrochloride, propoxyphene hydrochloride, propranolol hydrochloride, propulsid, propyl bis-acridone, propylhexedrine, propyliodone, propylthiouracil, proquazone, prorenoate potassium, proroxan hydrochloride, proscillaridin, prostalene, prostratin, protamine sulfate, protegrin, protirelin, protosufloxacin, protriptyline hydrochloride, proxazole, proxazole citrate, proxicromil, proxorphan tartrate, prulifloxacin, pseudoephedrine hydrochloride, desloratadine/pseudoephedrine sulfate (commercially available as CLARINEX-D® from Merck), puromycin, purpurins, pyrabrom, pyrantel, pamoate, pyrazinamide, pyrazofurin, pyrazoloacridine, pyridostigmine bromide, pyrilamine maleate, pyrimethamine, pyrinoline, pyrithione sodium, pyrithione zinc, pyrovalerone hydrochloride, pyroxamine maleate, pyrrocaine, pyrroliphene hydrochloride, pyrroinitrin, pyrvinium pamoate, quadazocine mesylate, quazepam, quazinone, quazodine, quazolast, quetiapine (commercially available as SEROQUEL® available from AstraZenica), quiflapon, quinagolide, quinaldine blue, quinapril, quinaprilat, quinazosin hydrochloride, quinbolone, quinctolate, quindecamine acetate, quindonium bromide, quinelorane hydrochloride, quinestrol, quinfamide, quingestanol acetate, quingestrone, quinidine gluconate, quinielorane hydrochloride, quinine sulfate, quinpirole hydrochloride, quinterenol sulfate, quinuclium bromide, quinupristin, quipazine maleate, rabeprazole sodium, racephenicol, racepinephrine, raf antagonists, rafoxanide, ralitoline, raloxifene, raltitrexed, ramatroban, ramipril, ramoplanin, ramosetron, ranelic acid, ranimycin, ranitidine, ranolazine, rauwolfia serpentina, recainam, recainam hydrochloride, reclazepam, regavirumab, regramostim, relaxin, relomycin, remacemide hydrochloride, remifentanil hydrochloride, remiprostol, remoxipride, repirinast, repromicin, reproterol hydrochloride, reserpine, resinferatoxin, resorcinol, retelliptine demethylated, reticulon, reviparin sodium, revizinone, rhenium re 186 etidronate, rhizoxin, ribaminol, ribavirin, riboprine, ribozymes, ricasetron, ridogrel, rifabutin, rifametane, rifamexil, rifamide, rifampin, rifapentine, rifaximin, retinamide, rilopirox, riluzole, rimantadine, rimcazole hydrochloride, rimexolone, rimiterol hydrobromide, rimoprogin, riodipine, rioprostil, ripazepam, ripisartan, risedronate sodium, risedronic acid, risocaine, risotilide hydrochloride, rispenzepine, risperdal, risperidone, ritanserin, ritipenem, ritodrine, ritolukast, ritonavir, rizatriptan benzoate, rocastine hydrochloride, rocuronium bromide, rodocaine, roflurane, rogletimide, rohitukine, rokitamycin, roletamicide, rolgamidine, rolicyprine, rolipram, rolitetracycline, rolodine, romazarit, romurtide, ronidazole, ropinirole (commercially available as REQUIP® from GlaxoSmithKline), ropitoin hydrochloride, ropivacaine, ropizine, roquinimex, rosaramicin, rosoxacin, rotoxamine, rosuvastatin (commercially available as CRESTOR® available from AstraZeneca), roxaitidine, roxarsone, roxindole, roxithromycin, rubiginone B1, ruboxyl, rufloxacin, rupatidine, rutamycin, ruzadolane, sabeluzole, safingol, safironil, saintopin, salbutamol, salcolex, salethamide maleate, salicyl alcohol, salicylamide, salicylate meglumine, salicylic acid, salmeterol, salnacediin, salsalate, sameridine, sampatrilat, sancycline, sanfetrinem, sanguinarium chloride, saperconazole, saprisartan, sapropterin, saquinavir, sarafloxacin hydrochloride, saralasin acetate, SarCNU, sarcophytol A, sargramostim, sarmoxicillin, sarpicillin, sarpogrelate, saruplase, saterinone, satigrel, satumomab pendetide, schick test control, scopafungin, scopolamine hydrobromide, scrazaipine hydrochloride, sdi 1 mimetics, secalciferol, secobarbital, seelzone, seglitide acetate, selegiline, selegiline hydrochloride, selenium sulfide, selenomethionine se 75, selfotel, sematilide, semduramicin, semotiadil, semustine, sense oligonucleotides, sepazonium chloride, seperidol hydrochloride, seprilose, seproxetine hydrochloride, seractide acetate, sergolexole maleate, serine, sermetacin, sermorelin acetate, sertaconazole, sertindole, sertraline, setiptiline, setoperone, sevirumab, sevoflurane, sezolamide, sibopirdine, sibutramine hydrochloride, signal transduction inhibitors, silandrone, sildenafil (commercially available as VIAGRA® from Pfizer Inc.), silipide, silteplase, silver nitrate, simendan, simtrazene, simvastatin (commercially available as ZOCOR® from Merck), sincalide, sinefungin, sinitrodil, sinnabidol, sipatrigine, sirolimus, sisomicin, sitogluside, sizofuran, sobuzoxane, sodium amylosulfate, sodium iodide I 123, sodium nitroprusside, sodium oxybate, sodium phenylacetate, sodium salicylate, solverol, solypertine tartrate, somalapor, somantadine hydrochloride, somatomedin B, somatomedin C, somatrem, somatropin, somenopor, somidobove, sonermin, sorbinil, sorivudine, sotalol, soterenol hydrochloride, sparfloxacin, sparfosate sodium, sparfosic acid, sparsomycin, sparteine sulfate, spectinomycin hydrochloride, spicamycin D, spiperone, spiradoline mesylate, spiramycin, spirapril hydrochloride, spiraprilat, spirogermanium hydrochloride, spiromustine, spironolactone, spiroplatin, spiroxasone, splenopentin, spongistatin 1, sprodiamide, squalamine, stallimycin hydrochloride, stannous pyrophosphate, stannous sulfur colloid, stanozolol, statolon, staurosporine, stavudine, steffimycin, stenbolone acetate, stepronin, stilbazium iodide, stilonium iodide, stipiamide, stiripentol, stobadine, streptomycin sulfate, streptonicozid, streptonigrin, streptozocin, stromelysin inhibitors, strontium chloride Sr 89, succibun, succimer, succinylcholine chloride, sucralfate, sucrosof ate potassium, sudoxicam, sufentanil, sufotidine, sulazepam, sulbactam pivoxil, sulconazole nitrate, sulfabenz, sulfabenzamide, sulfacetamide, sulfacytine, sulfadiazine, sulfadoxine, sulfalene, sulfamerazine, sulfameter, sulfamethazine, sulfamethizole, sulfamethoxazole, sulfamonomethoxine, sulfamoxole, sulfanilate zinc, sulfanitran, sulfasalazine, sulfasomizole, sulfazamet, sulfinalol hydrochloride, sulfinosine, sulfinpyrazone, sulfisoxazole, sulfomyxin, sulfonterol hydrochloride, sulfoxamine, sulinldac, sulmarin, sulnidazole, suloctidil, sulofenur, sulopenem, suloxifen oxalate, sulpiride, sulprostone, sultamicillin, sulthiame, sultopride, sulukast, sumarotene, sumatriptan, suncillin sodium, suproclone, suprofen, suradista, suramin, surfomer, suricamide maleate, suritozole, suronacrine maleate, suxemerid sulfate, swainsonine, symakalim, symclosene, symetine hydrochloride, synthetic glycosaminoglycans, tadalafil (commercially available as CIALIS® and ACIRCA® from from Eli Lilly and Company), taciamine hydrochloride, tacrine hydrochloride, tacrolimus, talampicillin hydrochloride, taleranol, talisomycin, tallimustine, talmetacin, talniflumate, talopram hydrochloride, talosalate, tametraline hydrochloride, tamoxifen (commercially available as NOLVADEX® from AstraZeneca), tampramine fumarate, tamsulosin hydrochloride, tandamine hydrochloride, tandospirone, tapgen, taprostene, tasosartan, tauromustine, taxane, taxoid, tazadolene succinate, tazanolast, tazarotene, tazifylline hydrochloride, tazobactam, tazofelone, tazolol hydrochloride, tebufelone, tebuquine, technetium Tc 99 m bicisate, teclozan, tecogalan sodium, teecleukin, teflurane, tegafur, tegretol, teicoplanin, telenzepine, tellurapyrylium, telmesteine, telmisartan, telomerase inhibitors, teloxantrone hydrochloride, teludipine hydrochloride, temafloxacin hydrochloride, tematropium methyl sulfate, temazepam, temelastine, temocapril, temocillin, temoporfin, temozolomide, tenofovir, tenidap, teniposide, tenosal, tenoxicam, tepirindole, tepoxalin, teprotide, terazosin, terbinafine, terbutaline sulfate (commercially available as BRICANYL® from AstraZeneca), terconazole, terfenadine, terflavoxate, terguride, teriparatide acetate, terlakiren, terlipressin, terodiline, teroxalene hydrochloride, teroxirone, tertatolol, tesicam, tesimide, testolactone, testosterone, tetracaine, tetrachlorodecaoxide, tetracycline, tetrahydrozoline hydrochloride, tetramisole hydrochloride, tetrazolast meglumine, tetrazomine, tetrofosmin, tetroquinone, tetroxoprim, tetrydamine, thaliblastine, thalidomide, theofibrate, theophylline, thiabendazole, thiamiprine, thiamphenicol, thiamylal, thiazesim hydrochloride, thiazinamium chloride, thiazolidinedione, thiethylperazine, thimerfonate sodium, thimerosal, thiocoraline, thiofedrine, thioguanine, thiomarinol, thiopental sodium, thioperamide, thioridazine, thiotepa, thiothixene, thiphenamil hydrochloride, thiphencillin potassium, thiram, thozalinone, threonine, thrombin, thrombopoietin, thrombopoietin mimetic, thymalfasin, thymopoietin receptor agonist, thymotrinan, thyromedan hydrochloride, thyroxine 1 125, thyroxine 1 131, tiacrilast, tiacrilast sodium, tiagabine, tiamenidine, tianeptine, tiapafant, tiapamil hydrochloride, tiaramide hydrochloride, tiazofurin, tibenelast sodium, tibolone, tibric acid, ticabesone propionate, ticarbodine, ticarcillin cresyl sodium, ticlatone, ticlopidine, ticrynafen, tienoxolol, tifurac sodium, tigemonam dicholine, tigestol, tiletamine hydrochloride, tilidine hydrochloride, tilisolol, tilnoprofen arbamel, tilorone hydrochloride, tiludronate disodium, tiludronic acid, timefurone, timobesone acetate, timolol, tin ethyl etiopurpurin, tinabinol, tinidazole, tinzaparin sodium, tioconazole, tiodazosin, tiodonium chloride, tioperidone hydrochloride, tiopinac, tiospirone hydrochloride, tiotidine, tiotropium bromide, tioxidazole, tipentosin hydrochloride, tipredane, tiprenolol hydrochloride, tiprinast meglumine, tipropidil hydrochloride, tiqueside, tiquinamide hydrochloride, tirandalydigin, tirapazamine, tirilazad, tirofiban, tiropramide, titanocene dichloride, tixanox, tixocortol pivalate, tizanidine hydrochloride, tobramycin, tocainide, tocamphyl, tofenacin hydrochloride, tolamolol, tolazamide, tolazoline hydrochloride, tolbutamide, tolcapone, tolciclate, tolfamide, tolgabide, lamotrigine, tolimidone, tolindate, tolmetin, tolnaftate, tolpovidone 1 131, tolpyrramide, tolrestat, tomelukast, tomoxetine hydrochloride, tonazocine mesylate, topiramate, topotecan, topotecan hydrochloride, topsentin, topterone, toquizine, torasemide, toremifene, torsemide, tosifen, tosufloxacin, totipotent stem cell factor, tracazolate, trafermin, tralonide, tramadol hydrochloride, tramazoline hydrochloride, trandolapril, tranexamic acid, tranilast, transcamide, translation inhibitors, trastuzumab (commercially available as HERCEPTIN® from Genentech), traxanox, trazodone hydrochloride, trazodone-hcl, trebenzomine hydrochloride, trefentanil hydrochloride, treloxinate, trepipam maleate, trestolone acetate, tretinoin, triacetin, triacetyluridine, triafungin, triamcinolone, triampyzine sulfate, triamterene, triazolam, tribenoside, tricaprilin, tricetamide, trichlormethiazide, trichohyalin, triciribine, tricitrates, triclofenol piperazine, triclofos sodium, triclonide, trientine, trifenagrel, triflavin, triflocin, triflubazam, triflumidate, trifluoperazine hydrochloride, trifluperidol, triflupromazine, triflupromazine hydrochloride, trifluridine, trihexyphenidyl hydrochloride, trilostane, trimazosin hydrochloride, trimegestone, trimeprazine tartrate, trimethadione, trimethaphan camsylate, trimethobenzamide hydrochloride, trimethoprim, trimetozine, trimetrexate, trimipramine, trimoprostil, trimoxamine hydrochloride, triolein 1 125, triolein 1 131, trioxifene mesylate, tripamide, tripelennamine hydrochloride, triprolidine hydrochloride, triptorelin, trisulfapyrimidines, troclosene potassium, troglitazone, trolamine, troleandomycin, trombodipine, trometamol, tropanserin hydrochloride, tropicamide, tropine ester, tropisetron, trospectomycin, trovafloxacin, trovirdine, tryptophan, tuberculin, tubocurarine chloride, tubulozole hydrochloride, tucarcsol, tulobuterol, turosteride, tybamate, tylogenin, tyropanoate sodium, tyrosine, tyrothricin, tyrphostins, ubenimex, uldazepam, undecylenic acid, uracil mustard, urapidil, urea, uredepa, uridine triphosphate, urofollitropin, urokinase, ursodiol, valaciclovir, valine, valnoctamide, valproate sodium, valproic acid, valsartan (commercially available as DIOVAN® from Novartis Pharmaceuticals), vamicamide, vanadeine, vancomycin, vaminolol, vapiprost hydrochloride, vapreotide, vardenafil (commercially available as LEVITRA® from GlaxoSmithKline), variolin B, vasopressin, vecuronium bromide, velaresol, velnacrine maleate, venlafaxine, veradoline hydrochloride, veramine, verapamil hydrochloride, verdins, verilopam hydrochloride, verlukast, verofylline, veroxan, verteporfin, vesnarinone, vexibinol, vidarabine, vigabatrin, viloxazine hydrochloride, vinblastine sulfate, vinburnine citrate, vincofos, vinconate, vincristine sulfate, vindesine, vindesine sulfate, vinepidine sulfate, vinglycinate sulfate, vinleurosine sulfate, vinorelbine, vinpocetine, vintoperol, vinxaltine, vinzolidine sulfate, viprostol, virginiamycin, viridofulvin, viroxime, vitaxin, volazocine, voriconazole, vorozole, voxergolide, warfarin sodium, xamoterol, xanomeline, xanoxate sodium, xanthinol niacinate, xemilofiban, xenalipin, xenbucin, xilobam, ximoprofen, xipamide, xorphanol mesylate, xylamidine tosylate, xylazine hydrochloride, xylometazoline hydrochloride, xylose, yangambin, zabicipril, zacopride, zafirlukast, zalcitabine, zaleplon, zalospirone, zaltidine hydrochloride, zaltoprofen, zanamivir, zankiren, zanoterone, zantac, zarirlukast, zatebradine, zatosetron, zatosetron maleate, zenarestat, zenazocine mesylate, zeniplatin, zeranol, zidometacin, zidovudine, zifrosilone, zilantel, zilascorb, zileuton, zimeldine hydrochloride, zinc undecylenate, zindotrine, zinoconazole hydrochloride, zinostatin, zinterol hydrochloride, zinviroxime, ziprasidone, zobolt, zofenopril calcium, zofenoprilat, zolamine hydrochloride, zolazepam hydrochloride, zoledronie acid, zolertine hydrochloride, zolmitriptan, zolpidem, zomepirac sodium, zometapine, zoniclezole hydrochloride, zonisamide, zopiclone, zopolrestat, zorbamyciin, zorubicin hydrochloride, zotepine, zucapsaicin, JTT-501 (PNU-182716) (reglitazar), AR-H039122, MCC-555 (netoglitazone), AR-H049020 (tesaglitazar), CS-011 (CI-1037), GW-409544x, KRP-297, RG-12525, BM-15.2054, CLX-0940, CLX-0921, DRF-2189, GW-1929, GW-9820, LR-90, LY-510929, NIP-221, NIP-223, JTP-20993, LY 29311 Na, FK 614, BMS 298585, R 483, TAK 559, DRF 2725 (ragaglitazar), L-686398, L-168049, L-805645, L-054852, demethyl asteriquinone B1 (L-783281), L-363586, KRP-297, P32/98, CRE-16336, EML-1625, pharmaceutically acceptable salts thereof (e.g., Zn, Fe, Mg, K, Na, F, CI, Br, I, acetate, diacetate, nitrate, nitrite, sulfate, sulfite, phosphate, and phosphite salts), pharmaceutically acceptable forms thereof with acid associates (e.g. HCl), and any combination thereof.

Porous implants of the present invention may, in some embodiments, comprise a plurality of polymeric particles sintered together at a plurality of contact points so as to form a porous network, at least one antioxidant, and optionally further comprise at least one growth factor, wherein the antioxidant and the growth factor are independently adsorbed to a surface of at least a portion of the polymeric particles and/or disposed in a pore of the porous network.

Growth factors may be useful in directing or enhancing cell growth on and/or through the porous implant. Examples of growth factors suitable for use in conjunction with the porous implants of the present invention may, in some embodiments, include, but are not limited to, adrenomedullin (AM), angiopoietin (Ang), bone morphogenetic proteins (BMPs), epidermal growth factor (EGF), erythropoietin (EPO), fibroblast growth factor (FGF), granulocyte colony-stimulating factor (G-CSF), granulocyte macrophage colony-stimulating factor (GM-CSF), hepatocyte growth factor (HGF), hepatoma-derived growth factor (HDGF), insulin-like growth factor (IGF), migration-stimulating factor, nerve growth factor (NGF) and other neurotrophins, platelet-derived growth factor (PDGF), transforming growth factor alpha (TGF-α), transforming growth factor beta (TGF-β), tumor_necrosis_factor-alpha (TNF-α), vascular endothelial growth factor (VEGF), interleukin-1 (“IL-1”), IL-2, IL-3, IL-4, IL-5, IL-6, IL-7, and the like, any derivative thereof, and any combination thereof.

Porous implants of the present invention may, in some embodiments, comprise a plurality of polymeric particles sintered together at a plurality of contact points so as to form a porous network, at least one antioxidant, and optionally further comprise at least one cell or cell-like structure, wherein the antioxidant and the cell or cell-like structure are independently adsorbed to a surface of at least a portion of the polymeric particles and/or disposed in a pore of the porous network.

Cells or cell-like structures may be useful in mitigating rejection of the porous implant and hastening patient recovery. Examples of cell or cell-like structure suitable for use in conjunction with the porous implants of the present invention may, in some embodiments, include, but are not limited to, endothelial cells, hepatic cells, myocytes, smooth muscle cells, nerve cells, progenitor cells, stem cells, parthenogenetic stem cell, activated version thereof (e.g., those overexpressing a marker), deactivated version thereof (e.g., those underexpressing a marker), synthetic cells, and the like. In some embodiments, cells may be from the patient, animal (e.g., pig), or another suitable donor.

Porous implants of the present invention may, in some embodiments, comprise a plurality of polymeric particles sintered together at a plurality of contact points so as to form a porous network, at least one antioxidant, and optionally further comprise at least one imaging agent, wherein the antioxidant and the imaging agent are independently adsorbed to a surface of at least a portion of the polymeric particles and/or disposed in a pore of the porous network. As used herein, the term “imaging agent” refers to a molecule, compound, or particle that interacts with electromagnetic radiation to enable one to ascertain an image.

Examples of imaging agents suitable for use in conjunction with the porous implants described herein may, in some embodiments, include, but are not limited to, magnetic resonance imaging agents (e.g., barium sulfate, iron oxide particles, gadolinium compounds, erbium compounds, gadolinium endofullerenes, and gadolinium endonanotubes), x-ray imaging agents (e.g., barium sulfate, iodine compounds, and iodine endonanotubes), ultrasound imaging agents (e.g., perfluorocarbons and air bubbles), near infrared imaging agents (e.g., carbon nanotubes, gold nanoparticles, silver nanoparticles, and gold nanoshells), bismuth compounds, tungsten compounds, and the like, and any combination thereof.

In some embodiments, imaging agents may be useful in monitoring the condition of the porous implants of the present invention (or the polymeric particles thereof) over the long-term. By way of nonlimiting example, an imaging agent disposed on the surface of the polymeric particles near the surface of the porous implant may be useful for noninvasively monitoring degradation (e.g., physical wear) of the porous implant.

Porous implants of the present invention may, in some embodiments, comprise a plurality of polymeric particles sintered together at a plurality of contact points so as to form a porous network, at least one antioxidant, and optionally further comprise at least one pigment and/or dye, wherein the antioxidant and the pigment and/or dye are independently adsorbed to a surface of at least a portion of the polymeric particles and/or disposed in a pore of the porous network. Examples of pigments and/or dyes suitable for use in conjunction with the porous implants described herein may, in some embodiments, include, but are not limited to, inorganic-based colorants, organic-based colorants, and the like, and any combination thereof.

In some embodiments, any of the foregoing additives (APIs, growth factors, cells and cell-like structures, imaging agents, pigments and/or dyes, and the like) may be independently included in the porous implants of the present invention in an amount ranging from a lower limit of about 0.001%, 0.01%, 0.05%, 0.1%, 0.5%, or 1% to an upper limit of about 10%_{,} 5%_{,} 3%_{,} 2%, or 1% by weight of the polymer, and wherein the amount may range from any lower limit to any upper limit and encompass any subset therebetween. The amount of each additive may depend on a number of factors that include, inter alia, the application of the porous implant, the composition of the antioxidant, the composition of the additive, and the composition of the polymeric particles utilized in conjunction with the porous implant.

In some embodiments, the porous implants of the present invention may comprise a porous network described herein, antioxidants, and optionally further comprise a reinforcing structure in contact with at least a portion of the porous network. In some embodiments, the porous network may be disposed on at least a portion of a surface of a reinforcing structure so as to leave a second portion of the reinforcing structure exposed to the local environment. In some embodiments, the porous network may be disposed about a reinforcing structure. It should be noted that as used herein, the term “exposed to the local environment” refers to being immediately exposed to a local environment (e.g., being able to be touched) as opposed to requiring the local environment to penetrate the porous network to access the object of interest. Exposure to the local environment may be achieved by at least one of protruding from the porous network, being disposed on the surface of the porous network, and any hybrid thereof.

Reinforcing structures may, in some embodiments, inter alia, enhance the mechanical strength of the porous implant of the present invention, enable post-production shaping of the porous implant of the present invention, enable securing of the porous implant to another object (e.g., a bone). In some embodiments, securing may be achieved, for example, with at least one of adhesives, screws, bone anchors, tacks, wires, sutures, stitches, and the like, any hybrid thereof, and any combination thereof.

Reinforcing structures suitable for use in conjunction with porous implants of the present invention may, in some embodiments, comprise metals (e.g., titanium), metal alloys (e.g., cobalt chromium alloys and trabecular metal), ceramics, and the like, and any combination thereof. Reinforcing structures suitable for use in conjunction with the porous implants of the present invention may, in some embodiments, be in the form of a rod, a tube, wire, mesh, a sheet, and the like, and any hybrid thereof.

In some embodiments, the porous implants of the present invention may comprise a porous network described herein, antioxidants, and optionally further comprise a skin layer disposed on at least a portion of a surface of the porous network. Skin layers may be advantageous as a smooth surface of the porous implant so as to minimize wear of the porous implant and/or abrasion of the adjacent tissue or bone. Skin layers suitable for use in conjunction with porous implants of the present invention may, in some embodiments, be in the form of a film, a mesh, a patch, a net, and the like.

Skin layers suitable for use in conjunction with porous implants of the present invention may, in some embodiments, comprise other polyethylenes (e.g., linear low density polyethylenes (LLDPE), low density polyethylenes (LDPE), and high density polyethylene (HDPE)), polypropylenes, ethylene-propylene copolymers, graft-modified olefin polymers, chlorinated polyethylenes, thermoplastic vulcanizates polyisoprenes, polyesters, polyamides, ethylene vinyl acetate copolymers, ethylene vinyl-methacrylate copolymers, silicones, polyethylene glycols, poly lactic acid, poly glycolic acid, polyethylene imine, polyurethanes, polyacrylonitrile, styrene block copolymers, rubbers, ethylene-carboxylic acid copolymers, ethylene acrylate copolymers, polybutylene, polybutadiene, nylons, polycarbonates, ethylene ethylacrylate polymers (EEA), ethylene styrene interpolymers (ESI), and the like, any derivative thereof, any copolymer thereof, and any combination thereof.

In some embodiments, the porous implants of the present invention may have any desired shape. As should be recognized by one of ordinary skill in the art, the desired shape may depend on, inter alia, the application in which the porous implants of the present invention are utilized.

In some embodiments, the porous implants of the present invention may be designed to be reshaped post-production (e.g., before implantation). Reshaping may be achieved by heating the porous implant and remodeling, generally by hand. Reshaping may further be achieved by cutting or the like. One skilled in the art with the benefit of this disclosure would understand that because the polymeric particles are sintered together, the ability to reshape the porous implant may be minimal, that is, allowing for minor adjustments in the shape so as to conform to the patient.

Porous implants described herein may, in some embodiments, comprise at least one plurality of polymeric particles (each independently having a characteristic or combination of characteristics described herein, e.g., polymer composition, polymer average molecular weight, shape, diameter in at least one dimension, surface roughness, bulk density, and any combination thereof) sintered together at a plurality of contact points so as to form a porous network (having a characteristic or combination of characteristics described herein, e.g., porosity, average diameter of pores, and any combination thereof) and at least one antioxidant described herein in an amount described herein, wherein the antioxidant is adsorbed to a surface of at least a portion of the polymeric particles and/or disposed in a pore of the porous network. In some embodiments, a porous implant described herein may optionally further comprise at least one of: at least one API described herein, at least one growth factor described herein, at least one cell or cell-like structure described herein, at least one imaging agent described herein, at least one pigment and/or dye described herein, each of which may independently be in an amount described herein, and wherein the antioxidant is adsorbed to a surface of at least a portion of the polymeric particles and/or disposed in a pore of the porous network. Further, in some embodiments, porous implants described herein may have any desired shape, may optionally further comprise reinforcing structures as described herein and/or skin layers as described herein, may optionally be designed to be reshaped post-production, or any combination thereof.

Porous implants of the present invention may, in some embodiments, be used as a craniomaxillofacial implant for implantation in the mouth, jaw, face, skull, nose, ear, chin, cheek, neck, ocular socket, cranium, larynx, lips, nasal, mandible, orbital, or associated structures.

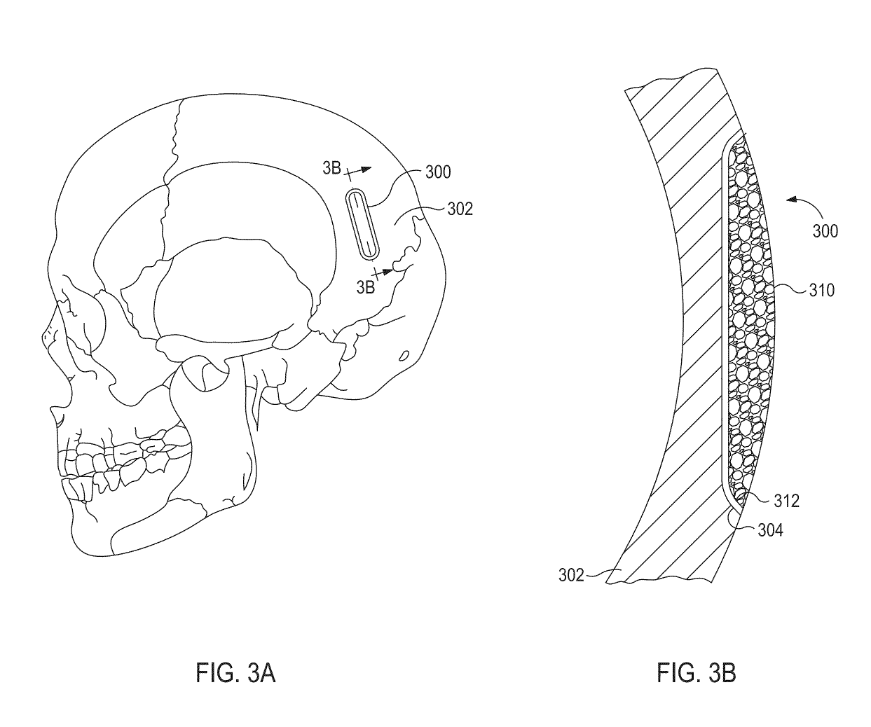

By way of a nonlimiting example illustrated in FIGS. 3A-B, a porous implant of the present invention may, in some embodiments, be a cranial implant 300 that comprises porous network 310 having skin layer 312 that is shaped to fit into cranial deformation 322 of cranium 302. As illustrated in FIG. 3B cranial implant 300 is secured in cranial deformation 304 with an adhesive disposed therebetween.

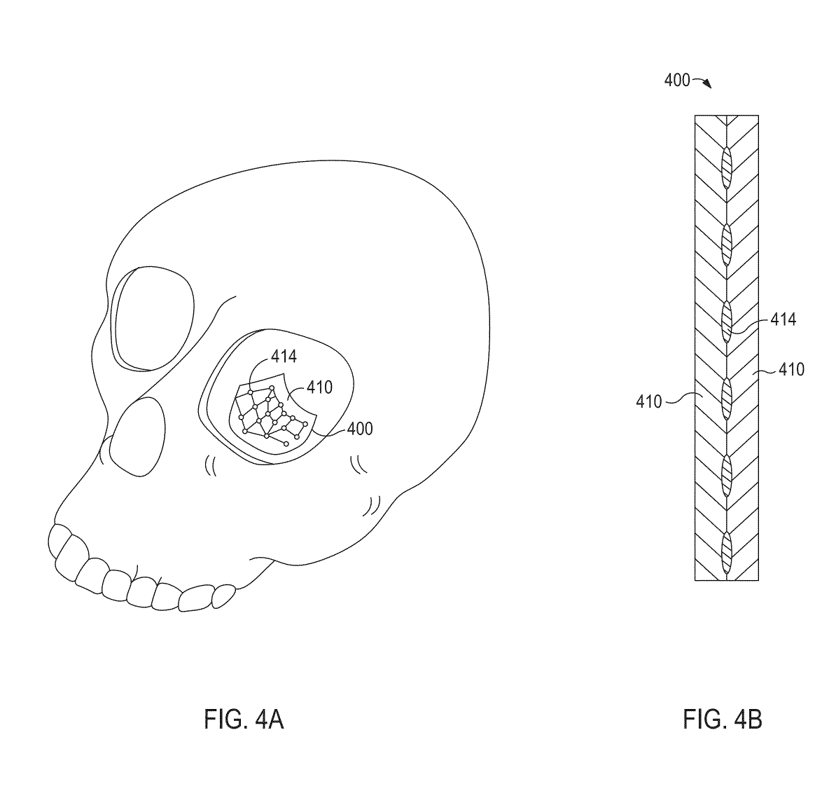

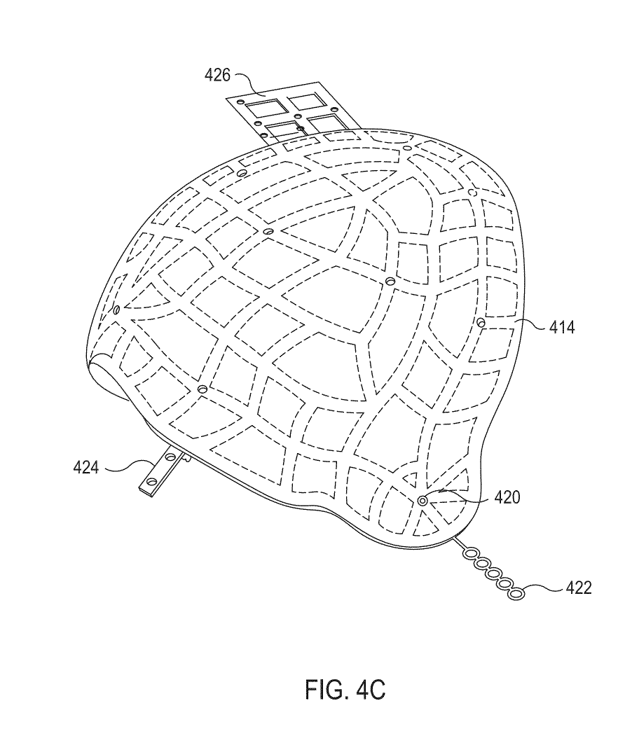

By way of another nonlimiting example illustrated in FIG. 4A, a porous implant of the present invention may, in some embodiments, be an orbital reconstruction implant 400. FIG. 4B illustrates an example cross-section of the orbital reconstruction implant 400 having a porous network 410 disposed about a reinforcing mesh 414. FIG. 4C illustrates an example of a reinforcing mesh 414 that includes several optional securing components 420, 422, 424, 428 that may be part of reinforcing mesh 414 as illustrated in securing component 420 or extend from and connect to reinforcing mesh 414 as illustrated in securing components 422, 424, 428.

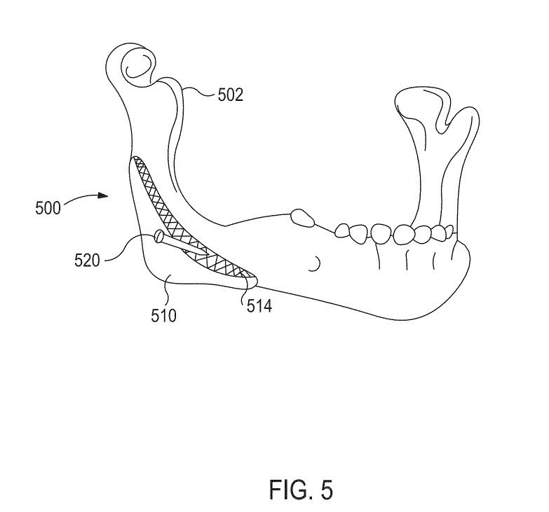

By way of yet another nonlimiting example illustrated in FIG. 5, porous implant of the present invention may, in some embodiments, be a mandible implant 500. As illustrated, mandible implant 500 comprises porous network 510 with structural plate 514 at the interface of the mandible implant 500 and the mandible 502. Further, mandible implant 500 is secured to the mandible 502 with screw 520 that extends through the porous network 510 and the structural plate 514.

As will be apparent to one of ordinary skill in the art, the above examples and corresponding figures are not limiting of the present invention as to, inter alia, the configurations of a porous implant and its potential implementation.

In some embodiments, forming porous implants of the present invention may involve heating a plurality of polymeric particles described herein in a mold so as to sinter together the polymeric particles at a plurality of contact points thereby forming a porous network; and contacting the polymeric particles with an antioxidant. In some embodiments, contacting may occur before and/or after heating.

In some embodiments, forming porous implants of the present invention may involve heating while applying pressure to a plurality of polymeric particles described herein in a mold so as to sinter together the polymeric particles at a plurality of contact points thereby forming a porous network; and contacting the polymeric particles with an antioxidant.

One skilled in the art with the benefit of this disclosure should understand that the temperature and pressure used in conjunction with forming porous networks described herein should be a combination to a temperature and a pressure suitable for fusing the polymeric particles at a plurality of contact points so as to form a porous network and not sinter the polymeric particles to the extent that a nonporous, solid mass is formed. One skilled in the art with the benefit of this disclosure should also recognize that the temperature and pressure used in forming porous networks described herein will be dependent on, inter alia, the geometry of the mold, the processing time, the composition of the polymeric particles, the composition of an antioxidant included during the molding process, the desired porosity of the porous network produced, and the like.

For example, temperatures suitable for use in conjunction with forming porous networks described herein may, in some embodiments, be at the softening temperature of the polymer of the polymeric particles, as appropriate. As used herein, the term “softening temperature” refers to the temperature above which a material becomes pliable, which is typically below the melting point of the material. In some embodiments, forming porous networks described herein may occur at temperatures ranging from a lower limit of about 90° C., 100° C., 110° C., 120° C., 130° C., 140° C., 150° C., 160° C., 170° C., or 180° C. or an upper limit of about 300° C., 275° C., 250° C., 225° C., 200° C., 175° C., or 150° C., and wherein the temperature may range from any lower limit to any upper limit and encompass any subset therebetween. It should be noted that forming methods that utilize increased pressure may, in some embodiments, enable forming porous networks described herein at lower temperatures.

In some embodiments, the polymeric particles may be preheated before introduction into the mold. Preheating may advantageously minimize the time to form the porous network. In some embodiments, the polymeric particles may be preheated to a temperature below the softening temperature of the polymer of the polymeric particles. In some embodiments, polymeric particles may be preheated to a temperature about 10%, about 5%, or about 1% below the softening temperature of the polymeric particles. In some embodiments, the polymeric particles may be preheated to a temperature about 10° C. below, about 5° C. below, or about 1° C. below the softening temperature of the polymeric particles.

In some embodiments, a reinforcing structure and/or a skin layer may be included in the mold when forming the porous network. In some embodiments, a reinforcing structure and/or a skin layer may be adhered to a porous network after having been formed.

In some embodiments, molds used in conjunction with forming porous networks described herein may be at least partially lined release agents. Release agents may advantageously ease removal of porous networks from the mold.

Release agents suitable for use in conjunction with forming porous networks described herein may be chemical release agents or physical release agents. Nonlimiting examples of chemical release agents may, in some embodiments, include, but are not limited to, oils, oil-based solutions and/or suspensions, soapy solutions and/or suspensions, coatings bonded to the mold surface, and the like, and any combination thereof. Nonlimiting examples of physical release agents may, in some embodiments, include, but are not limited to, papers, plastics, and the like, and any combination thereof.

In some embodiments, porous implants or a component thereof (e.g., the porous network or a reinforcing structure) may be cut and/or molded after production to yield a desired shape. Cutting and/or molding may occur, in some embodiments, at any suitable time, for example, before packaging or before implantation.

In some embodiments, forming porous implants of the present invention may involve doping the porous network after having been formed with antioxidants, additives described herein, and any combination thereof. In some embodiments, more than one doping procedure may be performed. In some embodiments, doping may occur while the porous network is in the mold. In some embodiments, doping may occur after the porous network has been removed from the mold.

It should be noted that doping includes, but is not limited to, applying, dipping, immersing, submerging, soaking, rinsing, washing, painting, coating, showering, drizzling, spraying, placing, dusting, sprinkling, affixing, and any combination thereof. Further, it should be noted that applying includes, but is not limited to, surface treatments, infusion treatments where the antioxidants and/or additives described herein become disposed in at least a portion of the pores of the porous network, or any combination thereof.

In some embodiments, doping may occur over a prolonged period of time. For example, doping a porous network with cells may occur over several days.

In some embodiments, a porous implant of the present invention may be implanted in and/or used in conjunction with the treatment of a patient. As used herein, the terms “subject” and “patient” are used interchangeably and refer to both human and nonhuman animals and insects. The term “nonhuman animals” as used herein includes all vertebrates, e.g., mammals and non-mammals, such as nonhuman primates, mice, rats, sheep, dogs, cats, horses, cows, chickens, amphibians, fish, reptiles, and the like. The term “insects” as used herein includes all arthropods, e.g., bees, flies, Drosophila flies, beetles, spiders, and the like.

In some embodiments, implantation into a patient may involve securing the porous implant to a bone.

In some embodiments, porous implants of the present invention may be included in a kit that also includes a set of instructions. In some embodiments, a kit may comprise a porous implant of the present invention and an article for securing the implant within the patient.

To facilitate a better understanding of the present invention, the following examples of preferred or representative embodiments are given. In no way should the following examples be read to limit, or to define, the scope of the invention.

### EXAMPLES

### Example 1

Several porous samples were prepared by heating various samples of UHMWPE particulates (as described in Table 1) while in a correspondingly sized mold. The physical properties of the resultant samples were analyzed and are reported in Table 2.

TABLE 1

| | Viscosity | | |
|---|---|---|---|
| | Number* | Melt Flow Index** | Average Particle |
| | (ml/g) | (g/10 min) | Size*** (microns) |
| UHMWPE-1 | 2250 | ~0 | 35 |
| UHMWPE-2 | 3800 | ~0 | 65 |
| UHMWPE-3 | 2100 | ~0 | 120 |
| UHMWPE-4 | 2250 | ~0 | 140 |
| UHMWPE-5 | 1000 | ~0 | 150 |
| UHMWPE-6 | 2200 | ~0 | 175 |
| UHMWPE-7 | 2000 | ~0 | 280 |
| UHMWPE-8 | 550 | 1.8 | 120 |
| UHMWPE-9 | 470 | 1.1 | 118 |
| UHMWPE-10 | 360 | 3.5 | 220 |

*As measured by DIN EN ISO 1628.
**As measured by EN ISO 1133 at 190° C./21.6 kg.
***As measured by laser diffraction.

TABLE 2

| | Pressure Drop* | Avg. Pore Size** | |
|---|---|---|---|
| | (mbar) | (microns) | Strength*** (MPa) |
| UHMWPE-1 | 30 | 20 | 1.7 |
| UHMWPE-2 | 75 | 15 | 2.5 |
| UHMWPE-3 | 40 | 25 | 2.5 |
| UHMWPE-4 | 10 | 52 | <1 |
| UHMWPE-5 | 20 | 40 | 4.5 |
| UHMWPE-6 | 20 | 33 | 1.8 |
| UHMWPE-7 | 8 | 60 | 2.3 |
| UHMWPE-8 | 20 | 40 | >6 |
| UHMWPE-9 | 20 | 50 | >8 |
| UHMWPE-10 | 15 | 130 | >8 |

*As measured using a sample of the porous article having a diameter of 140 mm, a width of 6.2-6.5 mm (depending on shrinkage), and an airflow rate of 7.5 m³/hour and measuring the drop in pressure in mbar across the width of the sample.
**As measured by Hg Intrusion DIN 133.
***As measured by DIN EN ISO 178.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

The invention claimed is:

1. A porous implant comprising:
a plurality of polymeric particles sintered together at a plurality of contact points to form a porous network having pores, the plurality of polymeric particles comprising polyethylene; and
an antioxidant disposed on a surface of at least some of the polymeric particles and/or in the pore of the porous network.

2. The porous implant of claim 1 further comprising a second plurality of polymeric particles comprising a polymer selected from the group consisting of a polyethylene-polypropylene copolymer, a polyethylene-polybutylene copolymer, polytetrafluoroethylene, and expanded polytetrafluoroethylene.

3. The porous implant of claim 1, wherein at least a plurality of the polymeric particles have a shape selected from the group consisting of spherical, ovular, substantially spherical, substantially ovular, prolate spheroidal, popcorn, substantially popcorn, potato, substantially potato, discus, platelet, flake, ligamental, acicular, fibrous, polygonal, randomly shaped, faceted, and any hybrid thereof, and the like with rounded corners and/or edges.

4. The porous implant of claim 1, wherein the polyethylene comprises at least one selected from the group consisting of high molecular weight polyethylene, very-high molecular weight polyethylene, ultrahigh molecular weight polyethylene, and any combination thereof.

5. The porous implant of claim 1, wherein the polymeric particles have a bulk density of about 0.10 g/cm³ to about 0.98 g/cm³.

6. The porous implant of claim 1, wherein the at least some of the polymeric particles have a diameter in at least one dimension ranging from about 10 microns to about 5000 microns.

7. The porous implant of claim 1, wherein the porous network comprises pores having a diameter of about 15 microns to about 500 microns.

8. The porous implant of claim 1, wherein the porous network has a porosity of about 40% to about 90%.

9. The porous implant of claim 1, wherein at least some of the polymeric particles have a surface that is about nano-rough to about micro-rough.

10. The porous implant of claim 1, wherein the antioxidant comprises at least one selected from the group consisting of anthocyanin, ascorbic acid, glutathione, lipoic acid, uric acid, resveratrol, flavonoids, carotenes, carotenoids, tocopherols, tocotrienols, ubiquinol, melatonin, secondary aromatic amines, benzofuranones, hindered phenols, polyphenols, and any combination thereof.

11. The porous implant of claim 1, wherein the antioxidant is present in an amount ranging from about 0.01% to about 10% by weight of the polyethylene.

12. The porous implant of claim 1 further comprising:
an additive disposed on a surface of at least some of the polymeric particles and/or disposed in a pore of the porous network, the additive being selected from the group consisting of active pharmaceutical ingredients, growth factors, cells or cell-like structures, imaging agents, pigments, dyes, and the like, and any combination thereof.

13. The porous implant of claim 1 further comprising:
a reinforcing structure in contact with at least a portion of the porous network.

14. The porous implant of claim 1 further comprising:
a skin layer disposed on at least a portion of a surface of the porous network.

15. The porous implant of claim 1, wherein at least a portion of the surgical implant is adapted to receive a surgical screw or surgical bone anchor.

16. A kit comprising the porous implant of claim 1 and a set of instructions.

17. A kit comprising the porous implant of claim 1 and a component capable of securing the porous implant to a bone.

18. A method comprising implanting the porous implant of claim 1 into a patient.

19. A porous implant comprising:
a plurality of polymeric particles sintered together at a plurality of contact points to form a porous network having pores, the plurality of polymeric particles comprising a polymer selected from the group consisting of a polyethylene-polypropylene copolymer, a polyethylene-polybutylene copolymer, polytetrafluoroethylene, and expanded polytetrafluoroethylene; and
an antioxidant disposed on a surface of at least some of the polymeric particles and/or in the pore of the porous network.

20. The porous implant of claim 19, wherein the at least one polymer comprises at least one selected from the group consisting of a high molecular weight polymer, a very-high molecular weight polymer, an ultrahigh molecular weight polymer, and any combination thereof.

21. The porous implant of claim 19, wherein the polymeric particles have a bulk density of about 0.10 g/cm³ to about 0.98 g/cm³.

22. The porous implant of claim 19, wherein the at least some of the polymeric particles have a diameter in at least one dimension ranging from about 10 microns to about 5000 microns.

23. The porous implant of claim 19, wherein the porous network comprises pores having a diameter of about 15 microns to about 500 microns.

24. The porous implant of claim 19, wherein the porous network has a porosity of about 40% to about 90%.

25. The porous implant of claim 19, wherein at least some of the polymeric particles have a surface that is about nano-rough to about micro-rough.

26. The porous implant of claim 19, wherein the antioxidant comprises at least one selected from the group consisting of anthocyanin, ascorbic acid, glutathione, lipoic acid, uric acid, resveratrol, flavonoids, carotenes, carotenoids, tocopherols, tocotrienols, ubiquinol, melatonin, secondary aromatic amines, benzofuranones, hindered phenols, polyphenols, and any combination thereof.

27. The porous implant of claim 19, wherein the antioxidant is present in an amount ranging from about 0.01% to about 10% by weight of the at least one polymer.

28. The porous implant of claim 19 further comprising:
an additive disposed on a surface of at least some of the polyethylene particles and/or disposed in a pore of the porous network, the additive being selected from the group consisting of active pharmaceutical ingredients, growth factors, cells or cell-like structures, imaging agents, pigments and/or dyes, and the like, and any combination thereof.

29. The porous implant of claim 19 further comprising:
a reinforcing structure in contact with at least a portion of the porous network.

30. The porous implant of claim 19 further comprising:
a skin layer disposed on at least a portion of a surface of the porous network.

31. The porous implant of claim 19, wherein at least a portion of the surgical implant is adapted to receive a surgical screw or surgical bone anchor.

32. A kit comprising the porous implant of claim 19 and a set of instructions.

33. A kit comprising the porous implant of claim 19 and a component capable of securing the porous implant to a bone.

34. A method comprising implanting the porous implant of claim 19 into a patient.

35. A method of forming a porous implant, the method comprising:
heating a plurality of polymeric particles in a mold so as to sinter together the polymeric particles at a plurality of contact points thereby forming a porous network, the plurality of polymeric particles comprising polyethylene; and
contacting the polymeric particles with an antioxidant.

36. The method of claim 35, wherein contacting occurs before heating.

37. The method of claim 35, wherein contacting occurs after heating.

38. The method of claim 35 further comprising:
placing a reinforcing structure in the mold such that polymeric particles are disposed about at least a portion of a surface of the reinforcing structure.

39. The method of claim 35 further comprising:
applying pressure during heating.

40. The method of claim 35, wherein heating is to a temperature between about 90° C. and about 300° C.

41. The method of claim 35 further comprising:
cutting the porous structure to a desired shape.

42. The method of claim 35 further comprising:
applying additional heat to the porous structure and reshaping the porous structure into a desired shape.

43. A method of forming a porous implant, the method comprising:
heating a plurality of polymeric particles in a mold so as to sinter together the polymeric particles at a plurality of contact points thereby forming a porous network, the plurality of polymeric particles comprising a polymer selected from the group consisting of a polyethylene-polypropylene copolymer, a polyethylene-polybutylene copolymer, polytetrafluoroethylene, and expanded polytetrafluoroethylene; and
contacting the polymeric particles with an antioxidant.

44. The method of claim 43, wherein contacting occurs before heating.

45. The method of claim 43, wherein contacting occurs after heating.

46. The method of claim 43 further comprising:
placing a reinforcing structure in the mold such that polymeric particles are disposed about at least a portion of a surface of the reinforcing structure.

47. The method of claim 43 further comprising:
applying pressure during heating.

48. The method of claim 43, wherein heating is to a temperature between about 90° C. and about 300° C.

49. The method of claim 43 further comprising:
cutting the porous structure to a desired shape.

50. The method of claim 43 further comprising:
applying additional heat to the porous structure and reshaping the porous structure into a desired shape.

51. A method for structurally modifying a portion of a cranium, the method comprising:
implanting a porous surgical implant into a cranium, the porous surgical implant comprising:
a porous structure formed from a plurality of polymeric particles sintered at a plurality of contact points, the plurality of polymeric particles comprising polyethylene, and
an antioxidant.

52. The method of claim 51 wherein the porous surgical implant further comprises a reinforcing structure.

53. The method of claim 51, where implanting involves securing the porous surgical implant to a bone.

54. A method for structurally modifying a portion of a cranium, the method comprising:
implanting a porous surgical implant into a cranium, the porous surgical implant comprising:
a porous structure formed from a plurality of polymeric particles sintered at a plurality of contact points, the plurality of polymeric particles comprising a polymer selected from the group consisting of a polyethylene-polypropylene copolymer, a polyethylene-polybutylene copolymer, polytetrafluoroethylene, and expanded polytetrafluoroethylene; and
an antioxidant.

55. The method of claim 54 wherein the porous surgical implant further comprises a reinforcing structure.

56. The method of claim 54, where implanting involves securing the porous surgical implant to a bone.
